SEQUENCE LISTING (14 sequences)

SEQ ID NO: 1 - DNA, 20 bp, Artificial Sequence
  Actin qPCR primer (forward)
atgatgatatcgccgcgctc

SEQ ID NO: 2 - DNA, 17 bp, Artificial Sequence
  Actin qPCR primer (reverse)
ccaccatcacgccctgg

SEQ ID NO: 3 - DNA, 20 bp, Artificial Sequence
  BRD1 qPCR primer (forward)
gaagaagcagtttgtggagc

SEQ ID NO: 4 - DNA, 20 bp, Artificial Sequence
  BRD1 qPCR primer (reverse)
gcagtctcagcgaagctcac

SEQ ID NO: 5 - DNA, 20 bp, Artificial Sequence
  BRD2 qPCR primer (forward)
gcttgggaagactttgttgg

SEQ ID NO: 6 - DNA, 20 bp, Artificial Sequence
  BRD2 qPCR primer (reverse)
tgtcagtcaccaggcagaag

SEQ ID NO: 7 - DNA, 22 bp, Artificial Sequence
  BRD3 qPCR primer (forward)
aagaagaaggacaaggagaagg

SEQ ID NO: 8 - DNA, 20 bp, Artificial Sequence
  BRD3 qPCR primer (reverse)
cttcttggcaggagccttct

SEQ ID NO: 9 - DNA, 22 bp, Artificial Sequence
  BRD4 qPCR primer (forward)
caggagggttgtacttatagca

SEQ ID NO: 10 - DNA, 22 bp, Artificial Sequence
  BRD4 qPCR primer (reverse)
ctactgtgacatcatcaagcac

SEQ ID NO: 11 - DNA, 20 bp, Artificial Sequence
  BRDT qPCR primer (forward)
tcaaagatcccgattgaacc

SEQ ID NO: 12 - DNA, 20 bp, Artificial Sequence
  BRDT qPCR primer (reverse)
cggaaaggtacttgggacaa

SEQ ID NO: 13 - DNA, 20 bp, Artificial Sequence
  RFP specific primer (forward)
cggttaaaggtgccgtctcg

SEQ ID NO: 14 - DNA, 20 bp, Artificial Sequence
  RFT specific primer (reverse)
aggcttcccaggtcacgatg

(19) United States
(12) Patent Application Publication
(10) Pub. No.: US 2019/0262355 A1
(43) Pub. Date: Aug. 29, 2019

(54) COMBINATION OF A BRD4 INHIBITOR AND AN ANTIFOLATE FOR THE THERAPY OF CANCER

(71) Applicant: CeMM-Forschungszentrum für Molekulare Medizin GmbH, Wien (AT)

(72) Inventors: Sara SDELCI, Wien (AT); Stefan KUBICEK, Wien (AT)

(21) Appl. No.: 16/349,552

(22) PCT Filed: Nov. 14, 2017
(86) PCT No.: PCT/EP2017/079225

§ 371 Date: May 13, 2019

(30) Foreign Application Priority Data
Nov. 14, 2016 (EP) 16198760.7

(51) Int. Cl. A61K 31/551; A61P 35/00; A61K 31/519; G01N 33/574; C12Q 1/6886

(52) U.S. Cl. CPC A61K 31/551; A61P 35/00; C12Q 2600/106; G01N 33/574; C12Q 1/6886; A61K 31/519

(57) ABSTRACT

The present invention relates to the combination of a BRD4 inhibitor with an antifolate (particularly an MTHFD1 inhibitor) for use in the treatment or prevention of cancer. The invention also relates to an antifolate (particularly an MTHFD1 inhibitor) for use in resensitizing a BRD4 inhibitor-resistant cancer to the treatment with a BRD4 inhibitor. The invention further provides a pharmaceutical composition comprising a BRD4 inhibitor, an antifolate (particularly an MTHFD1 inhibitor), and a pharmaceutically acceptable excipient. Moreover, the invention provides a method of assessing the susceptibility or responsiveness of a subject to the treatment with a BRD4 inhibitor, wherein the subject has been diagnosed as suffering from cancer or is suspected of suffering from cancer, the method comprising determining the level of nuclear folate and/or the level of expression of MTHFD1 in a sample obtained from the subject.

The present invention relates to the combination of a BRD4 inhibitor with an antifolate (particularly an MTHFD1 inhibitor) for use in the treatment or prevention of cancer. The invention also relates to an antifolate (particularly an MTHFD1 inhibitor) for use in resensitizing a BRD4 inhibitor-resistant cancer to the treatment with a BRD4 inhibitor. The invention further provides a pharmaceutical composition comprising a BRD4 inhibitor, an antifolate (particularly an MTHFD1 inhibitor), and a pharmaceutically acceptable excipient. Moreover, the invention provides a method of assessing the susceptibility or responsiveness of a subject to the treatment with a BRD4 inhibitor, wherein the subject has been diagnosed as suffering from cancer or is suspected of suffering from cancer, the method comprising determining the level of nuclear folate and/or the level of expression of MTHFD1 in a sample obtained from the subject.

Chromatin controls gene expression in response to environmental signals. Key mediators of this process are cellular metabolites that act as cofactors and inhibitors of chromatin-modifying enzymes and are thought to enter the nucleus through uncontrolled influx from the cytoplasm.

Bromodomain-containing protein 4 (BRD4) is an important chromatin regulator, with described roles in gene activation, DNA damage, cell proliferation and cancer progression¹⁻⁸. At least seven inhibitors of this bromodomain protein have reached the clinical stage and are currently evaluated for their efficacy in different cancers. The clinical benefit of BRD4 inhibitors is largely considered to be mediated by the direct repression of the driver oncogene c-MYC^{2,7}. This notion is further supported by the recent discovery of the restoration of MYC expression and activation of WNT signaling as the major resistance mechanism to BRD4 inhibitors^{9,10}.

Despite its clinical importance and the broad role of BRD4 in chromatin organization, surprisingly little is known about factors that are directly required for BRD4 function. The focus of most studies is the role of BRD4 as transcriptional activator, thought to be mediated by the binding of the tandem bromodomains to acetylated histone lysines, resulting in transcription factor recruitment and pTEFb mediated activation of paused RNA polymerase II. In addition, several proteins have been identified as direct BRD4 interactors, including viral protein LANA-1¹¹ and chromatin proteins NSD3, ATAD5, CHD4, LTSCR1, and JMJD5¹²⁻¹⁴.

To identify if these or other proteins are directly required for BRD4 function, the inventors made use of a reporter cell line for monitoring the inhibition of BRD4. They recently established the REDS (reporter for epigenetic drug screening) cell line, confirmed the high selectivity of the reporter system for functional BRD4 inhibition and successfully pinpointed a crosstalk of BRD4 and TAF1 bromodomain inhibitors¹⁵. The haploid nature of the KBM7 cell line employed for the generation of REDSs makes it ideally suited for genetic screens for new BRD4 functional partners using a Gene-Trap (GT) approach. Here, remarkable results have been obtained with this strategy, leading to the identification of methylenetetrahydrofolate dehydrogenase 1 (MTHFD1) as genetic and physical interactor of BRD4. The description of a nuclear role of this C-1-tetrahydrofolate synthase, as identified in the present invention, highlights a robust connection between cancer epigenetics and folate metabolism.

As detailed in the examples, the inventors found a direct transcriptional role of the folate-pathway enzyme MTHFD1, which they identified from a haploid genetic screen for factors required for BRD4 function. It has been shown that MTHFD1 can translocate into the nucleus and a fraction of it is chromatin-bound via direct physical interaction with BRD4, and occupies a subset of BRD4-bound loci in the genome. Moreover, it has been shown in multiple cell lines that the inhibition or downregulation of MTHFD1 induces similar transcriptional changes as inhibition or downregulation of BRD4. It has furthermore been demonstrated that the inhibition of either BRD4 or MTHFD1 results in similar changes in the nuclear metabolite composition. Moreover, it has been found that pharmacologic inhibitors of the two enzymes synergize, and that methotrexate can render (S)-JQ1 resistant cells sensitive. In addition, pharmacologic inhibitors of the two enzymes also synergize in vivo arresting tumor proliferation in a mouse xenograft model. Finally, the finding that the majority of biosynthetic enzymes required for nucleotide biosynthesis are in a tightly chromatin-bound fraction indicates a direct role of nuclear metabolism in the control of gene expression and enables new clinical strategies for BRD4 inhibitors in cancer.

Accordingly, the inventors have identified MTHFD1 as a functional genetic interactor of BRD4 and have shown that the loss of MTHFD1 phenocopies BRD4 inhibition. MTHFD1 is a key enzyme in folate metabolism, thereby providing important intermediates for the biosynthesis of nucleotides and methionine. MTHFD1 and BRD4 interact physically in the nucleus, and inhibition of either protein causes similar changes to nuclear metabolite composition. Inhibitors of the two enzymes have been found to synergize to impair the viability of multiple cancer cell lines.

Thus, in the context of the present invention, it has surprisingly been found that the use of a BRD4 inhibitor (such as, e.g., (S)-JQ1) in combination with an antifolate (particularly an MTHFD1 inhibitor; such as, e.g., methotrexate) provides a synergistically enhanced therapeutic effect against a range of different cancer cell lines, and hence allows an improved therapy of cancer. Moreover, it has been found that an antifolate (particularly an MTHFD1 inhibitor, such as methotrexate) can be used to resensitize BRD4 inhibitor-resistant cancer (such as (S)-JQ1-resistant cancer) to the treatment with a BRD4 inhibitor. The combined use of a BRD4 inhibitor together with an antifolate (or an MTHFD1 inhibitor) is furthermore advantageous as it allows to prevent or reduce the emergence of resistance to BRD4 inhibitors in cancer. The present invention thus solves the problem of providing an improved therapy for cancer, including in particular BRD4 inhibitor-resistant cancer,

Accordingly, the present invention provides a combination of a BRD4 inhibitor and an antifolate (particularly a combination of a BRD4 inhibitor and an MTHFD1 inhibitor) for use in therapy, preferably for use in treating or preventing cancer.

The invention also provides a BRD4 inhibitor for use in therapy, preferably for use in treating or preventing cancer, wherein the BRD4 inhibitor is to be administered in combination with an antifolate (particularly an MTHFD1 inhibitor).

The invention likewise relates to an antifolate (particularly an MTHFD1 inhibitor) for use in therapy, preferably for use in treating or preventing cancer, wherein the antifolate (or the MTHFD1 inhibitor) is to be administered in combination with a BRD4 inhibitor.

The invention further provides a pharmaceutical composition comprising a BRD4 inhibitor, an antifolate (particularly an MTHFD1 inhibitor), and a pharmaceutically acceptable excipient. The invention also relates to the aforementioned pharmaceutical composition for use in treating or preventing cancer.

Moreover, the present invention provides an antifolate (particularly an MTHFD1 inhibitor) for use in resensitizing a BRD4 inhibitor-resistant cancer to the treatment with a BRD4 inhibitor. The BRD4 inhibitor-resistant cancer may, in particular, be a cancer that is resistant to BRD4 inhibitor monotherapy.

The present invention furthermore relates to the use of a BRD4 inhibitor in combination with an antifolate (particularly an MTHFD1 inhibitor) for the preparation of a medicament for treating or preventing cancer. The invention likewise provides the use of a BRD4 inhibitor for the preparation of a medicament for treating or preventing cancer, wherein the BRD4 inhibitor is to be administered in combination with an antifolate (particularly an MTHFD1 inhibitor). The invention also relates to the use of an antifolate (particularly an MTHFD1 inhibitor) for the preparation of a medicament for treating or preventing cancer, wherein the antifolate (or the MTHFD1 inhibitor) is to be administered in combination with a BRD4 inhibitor. Moreover, the invention refers to the use of an antifolate (particularly an MTHFD1 inhibitor) for the preparation of a medicament for resensitizing a BRD4 inhibitor-resistant cancer (particularly a cancer that is resistant to BRD4 inhibitor monotherapy) to the treatment with a BRD4 inhibitor.

The present invention likewise relates to a method of treating or preventing a disease or disorder, preferably cancer, the method comprising administering a BRD4 inhibitor in combination with an antifolate (particularly an MTHFD1 inhibitor) to a subject (e.g., a human) in need thereof. The invention further provides a method of resensitizing a BRD4 inhibitor-resistant cancer to the treatment with a BRD4 inhibitor, the method comprising administering an antifolate (particularly an MTHFD1 inhibitor) to a subject (e.g., a human) in need thereof.

As described above, the present invention relates to the combination of a BRD4 inhibitor with an antifolate (particularly an MTHFD1 inhibitor) for use in therapy, preferably for use in treating or preventing cancer. The BRD4 inhibitor and the antifolate (or the BRD4 inhibitor and the MTHFD1 inhibitor) can be provided in separate pharmaceutical formulations. Such separate formulations can be administered either simultaneously or sequentially (e.g., the formulation comprising the BRD4 inhibitor may be administered first, followed by the administration of the formulation comprising the antifolate (or the MTHFD1 inhibitor), or vice versa). However, the BRD4 inhibitor and the antifolate (or the BRD4 inhibitor and the MTHFD1 inhibitor) can also be provided in a single pharmaceutical formulation. Accordingly, the invention also relates to a pharmaceutical composition comprising a BRD4 inhibitor, an antifolate (particularly an MTHFD1 inhibitor), and a pharmaceutically acceptable excipient. This novel pharmaceutical composition is useful, in particular, for the treatment or prevention of cancer.

The disease/disorder to be treated or prevented in accordance with the present invention is preferably a hyperproliferative disorder, and most preferably cancer. The cancer to be treated or prevented may, for example, be selected from gastrointestinal cancer, colorectal cancer, liver cancer (e,g., hepatocellular carcinoma), pancreatic cancer, stomach cancer, genitourinary cancer, bladder cancer, biliary tract cancer, testicular cancer, cervical cancer, malignant mesothelioma, esophageal cancer, laryngeal cancer, prostate cancer (e.g., hormone-refractory prostate cancer), lung cancer (e.g., small cell lung cancer or non-small cell lung cancer), breast cancer (e.g., triple-negative breast cancer, or breast cancer having a BRCA1 and/or BRCA2 gene mutation), hematological cancer, leukemia (e.g., acute lymphoblastic leukemia, acute myeloid leukemia, chronic lymphocytic leukemia, or chronic myeloid leukemia), lymphoma (e.g., Hodgkin lymphoma or non-Hodgkin lymphoma, such as, e.g., follicular lymphoma or diffuse large B-cell lymphoma), multiple myeloma, ovarian cancer, brain cancer, neuroblastoma, Ewing's sarcoma, osteogenic sarcoma, kidney cancer, epidermoid cancer, skin cancer, melanoma, head and/or neck cancer (e.g., head and neck squamous cell carcinoma), and mouth cancer. Preferably, the cancer to be treated or prevented is selected from prostate cancer, breast cancer, acute myeloid leukemia, acute lymphocytic leukemia, non-Hodgkin's lymphoma, multiple myeloma, bladder cancer, head and neck cancer, glioblastoma, mesothelioma, osteogenic sarcoma, choriocarcinoma, and NUT midline carcinoma. It is particularly preferred that the cancer to be treated or prevented (including any of the above-mentioned specific types of cancer) is a BRD4-dependent cancer and/or c-MYC-dependent cancer.

As described above, the present invention also relates to the treatment of BRD4 inhibitor-resistant cancer using the drug combination of the invention, i.e. a BRD4 inhibitor in combination with an antifolate (particularly an MTHFD1 inhibitor). The cancer to be treated (including any of the specific types of cancer referred to in the preceding paragraph) may thus also be a BRD4 inhibitor-resistant cancer, particularly a cancer that is resistant to BRD4 inhibitor monotherapy.

The BRD4 inhibitor to be used in accordance with the present invention is not particularly limited, and is preferably any one of (S)-JQ1, CeMMEC2, I-BET 151 (or GSK1210151A), I-BET 762 (or GSK525762), PF-1, bromosporine, OTX-015, TEN-010, CPI-203, CPI-0610, RVX-208, BI2536, TG101348, LY294002, or a pharmaceutically acceptable salt or solvate of any of these agents. These compounds are commercially available and/or their synthesis is described in the literature. For example, the compound CeMMEC2 can be obtained from AKos GmbH (Steinen, Germany). The BRD4 inhibitor may also be any one of the compounds disclosed in WO 2012/174487, WO 20141076146, US 2014/0135336, WO 2014/134583, WO 2014/191894, WO 2014/191896, US 2014/0349990, or WO 2014/191906. It is particularly preferred that the BRD4 inhibitor is (S)-JQ1 or CeMMEC2, and even more preferably it is (S)-JQ1.

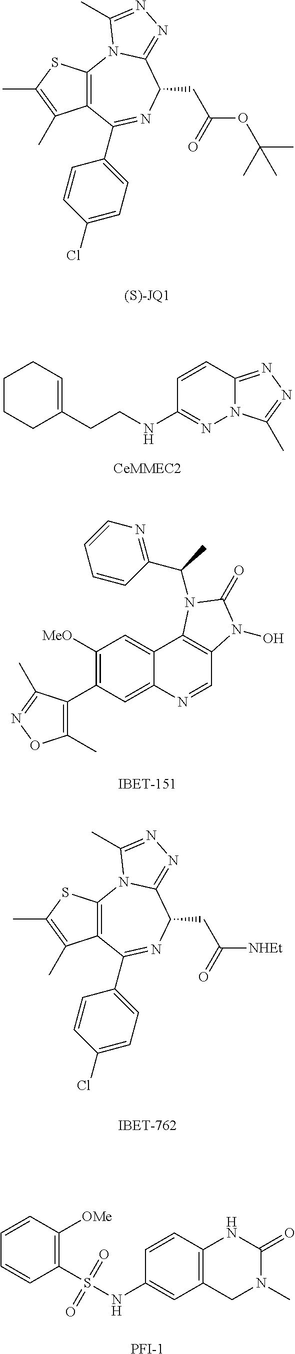

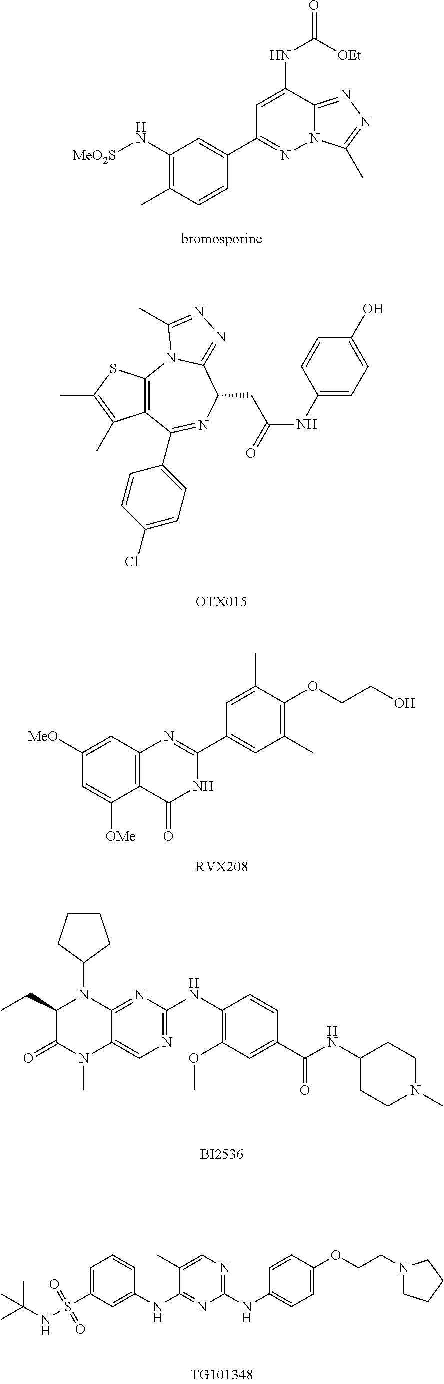

Antifolates constitute an established class of pharmacological agents that antagonize or block the effects of folic acid on cellular processes. Antifolates like methotrexate and pemetrexed are approved agents used in cancer chemotherapy; they primarily target DHFR, but have also been shown to inhibit other enzymes in folate metabolism including MTHFD1. The antifolate to be used in accordance with the present invention is preferably an MTHFD1 inhibitor, i.e. an inhibitor of methylenetetrahydrofolate dehydrogenase 1 (MTHFD1). Examples of the antifolate include, in particular, methotrexate, pemetrexed, trimetrexate, edatrexate, lometrexol, 5-fluorouracil, pralatrexate, aminopterin, and pharmaceutically acceptable salts and solvates of these agents. A particularly preferred antifolate (or MTHFD1 inhibitor) in accordance with the invention is methotrexate or a pharmaceutically acceptable salt or solvate thereof (e.g., methotrexate sodium).

The scope of the invention embraces all pharmaceutically acceptable salt forms of the compounds to be used in accordance with the invention (also referred to as the compounds of the drug combination provided herein; including in particular the BRD4 inhibitors, the antifolates, and the MTHFD1 inhibitors referred to in this specification), which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesuifonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts.

Moreover, the scope of the invention embraces the compounds to be used in accordance with the invention in any solvated form, including, e.g., solvates with water (i.e., as a hydrate) or solvates with organic solvents such as, e.g., methanol, ethanol or acetonitrile (i.e., as a methanolate, ethanolate or acetonitrilate), or in any crystalline form (i.e., as any polymorph), or in amorphous form. It is to be understood that such solvates of the compounds to be used in accordance with the invention also include solvates of pharmaceutically acceptable salts of the respective compounds.

Furthermore, the compounds to be used in accordance with the invention may exist in the form of different isomers, in particular stereoisomers (including, e.g., geometric isomers (or cis/trans isomers), enantiomers and diastereomers) or tautomers. All such isomers of the compounds referred to in this specification are contemplated as being part of the present invention, either in admixture or in pure or substantially pure form, As for stereoisomers, the invention embraces the isolated optical isomers of the compounds to be used according to the present invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds provided herein.

The scope of the invention also embraces the compounds to be used in accordance with the invention, in which one or more atoms are replaced by a specific isotope of the corresponding atom. For example, the invention encompasses the use of the compounds referred to in this specification, in which one or more hydrogen atoms (or, e.g., all hydrogen atoms) are replaced by deuterium atoms (i.e., ²H; also referred to as “D”). Accordingly, the invention also embraces the compounds to be used in accordance with the invention which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds to be used in accordance with the invention can be increased using deuteration techniques known in the art. For example, a compound referred to in the present specification or a reactant or precursor to be used in the synthesis of the corresponding compound can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy, Unless specifically indicated otherwise, it is preferred that the compounds to be used in accordance with the invention are not enriched in deuterium. Accordingly, the presence of naturally occurring hydrogen atoms or ¹H hydrogen atoms in the compounds to be used in accordance with the invention is preferred.

The invention furthermore provides a method (particularly an in vitro method) of assessing the susceptibility or responsiveness of a subject to the treatment with a BRD4 inhibitor, wherein the subject has been diagnosed as suffering from cancer or is suspected of suffering from cancer, the method comprising determining the level of nuclear folate and/or the level of expression of MTHFD1 in a sample obtained from the subject. It has been found that a smaller/lower level of nuclear folate and/or a smaller/lower expression level of MTHFD1, particularly a smaller/lower level of MTHFD1 protein in the nucleus of the corresponding cell, correlates with a greater susceptibility/responsiveness of the subject to the treatment with a BRD4 inhibitor. While the total expression level of MTHFD1 can also be predictive, the amount of MTHFD1 protein in the nucleus allows an even more accurate assessment of the susceptibility/responsiveness of the subject to the treatment with a BRD4 inhibitor. It is thus preferred that the level of expression of MTHFD1 is determined by determining the level of nuclear MTHFD1 protein, i.e., the amount of MTHFD1 protein in the nucleus of the corresponding cells.

The invention further provides a method (particularly an in vitro method) of assessing the susceptibility or responsiveness of a subject to the treatment with a BRD4 inhibitor, wherein the subject has been diagnosed as suffering from cancer or is suspected of suffering from cancer, the method comprising a step of determining the level of nuclear folate and/or the level of expression of MTHFD1 in a sample obtained from the subject, wherein a smaller level of nuclear folate and/or a smaller expression level of MTHFD1 in the sample from the subject is/are indicative of the subject being more susceptible or more responsive to the treatment with a BRD4 inhibitor. In this method, the level of nuclear folate (i.e., the level of folate in the nucleus of the corresponding cells), or the level of expression of MTHFD1, or both can be determined in order to assess the susceptibility or responsiveness of the subject to the treatment with a BRD4 inhibitor.

Accordingly, the invention also relates to a method (particularly an in vitro method) of assessing the susceptibility or responsiveness of a subject to the treatment with a BRD4 inhibitor, wherein the subject has been diagnosed as suffering from cancer or is suspected of suffering from cancer, the method comprising a step of determining the level of nuclear folate in a sample obtained from the subject, wherein a smaller level of nuclear folate in the sample from the subject is indicative of the subject being more susceptible or more responsive to the treatment with a BRD4 inhibitor.

The invention further relates to a method (particularly an in vitro method) of assessing the susceptibility or responsiveness of a subject to the treatment with a BRD4 inhibitor, wherein the subject has been diagnosed as suffering from cancer or is suspected of suffering from cancer, the method comprising a step of determining the level of expression of MTHFD1 in a sample obtained from the subject, wherein a smaller expression level of MTHFD1 in the sample from the subject is indicative of the subject being more susceptible or more responsive to the treatment with a BRD4 inhibitor. The level of expression of MTHFD1 is preferably determined by determining the level of nuclear MTHFD1 protein.

The description of exemplary or preferred features/embodiments provided herein with respect to the combination of a BRD4 inhibitor with an antifolate (or an MTHFD1 inhibitor), including inter alia the description of the cancer, the BRD4 inhibitor and the subject/patient, also applies to the above-described methods.

The sample to be used in the above-described methods is preferably a cancer tissue biopsy sample. Depending on the specific type of cancer, the sample may also be a body fluid, such as a blood sample (e.g., a whole blood sample, or a peripheral blood mononuclear cell fraction).

In some of the methods described above, the level of expression of MTHFD1 is determined in a sample obtained from the subject to be examined. The level of expression can be determined, for example, by determining the level of translation or the level of transcription of MTHFD1. Thus, the amount of MTHFD1 protein in the sample can be determined or the amount of MTHFD1 mRNA in the sample can be established in order to determine the level of expression of MTHFD1. This can be accomplished using methods known in the art, as described, e.g., in Green et al., 2012 (i.e., Green, M R et al., Molecular Cloning: A Laboratory Manual, Cold Spring Harbor Laboratory Press, Fourth Edition, 2012, ISBN: 978-1936113422). Preferably, the level of expression of MTHFD1 is determined by determining the level of translation of MTHFD1. More preferably, the level of expression of MTHFD1 is determined by determining the level of nuclear MTHFD1 protein, i.e. the amount of MTHFD1 protein specifically in the nucleus of the corresponding cells.

The level of translation of MTHFD1 can, e.g., be determined using antibody-based assays, such as an immunohistochemical method, an enzyme-linked immunosorbent assay (ELISA) or a radioimmunoassay (RIA), wherein antibodies directed specifically against the MTHFD1 protein to be quantified are employed, or mass spectrometry, a gel-based or blot-based assay, or flow cytometry (e.g., FACS). If the level of translation is to be determined, it may be advantageous to include one or more protease inhibitors in the sample from the subject.

The level of transcription of MTHFD1 can, e.g., be determined using a quantitative (real-time) reverse transcriptase polymerase chain reaction (“qRT-PCR”) or using a microarray (see, e.g., Ding C, et al. J Biochem Mol Biol. 2004; 37(1):1-10). It is also possible to use single-cell gene expression analysis techniques, such as single-cell qRT-PCR or single-cell microarray analysis, in order to determine the level of transcription of MTHFD1 in single cells from the sample. If the level of transcription is to be determined, it may further be advantageous to include one or more RNase inhibitors in the sample from the subject.

In accordance with the present invention, it is preferred that the level of expression of MTHFD1 is determined by determining the level of translation of MTHFD1, and particularly by determining the level of nuclear MTHFD1 protein. Preferably, the level of translation of MTHFD1 (or the level of nuclear MTHFD1 protein) is determined using an antibody-based assay, mass spectrometry, a gel-based or blot-based assay, or flow cytometry, more preferably using an immunohistochemical method, an enzyme-linked immunosorbent assay, or a radioimmunoassay, even more preferably using an immunohistochemical method. Methods for immunohistochemical staining are well-known in the art and are described, e.g., in: Renshaw, S., Immunohistochemistry: Methods Express, Scion Publishing Ltd, Bloxham (UK), 2007, ISBN: 9781904842033 (particularly chapter 4 “Immunochemical staining techniques”); Key, M., lmmunohistochemical staining methods: education guide, 2006 (particularly chapter 9); and Chen, X. et al. N Am J Med Sci 2(5), 241-245 (2010).

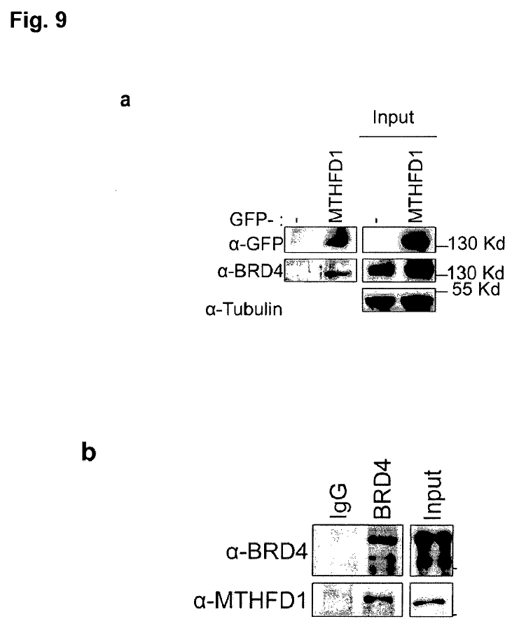

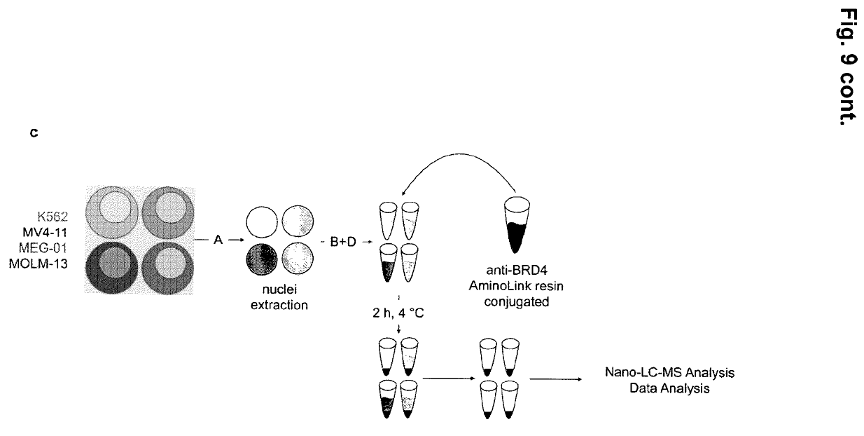

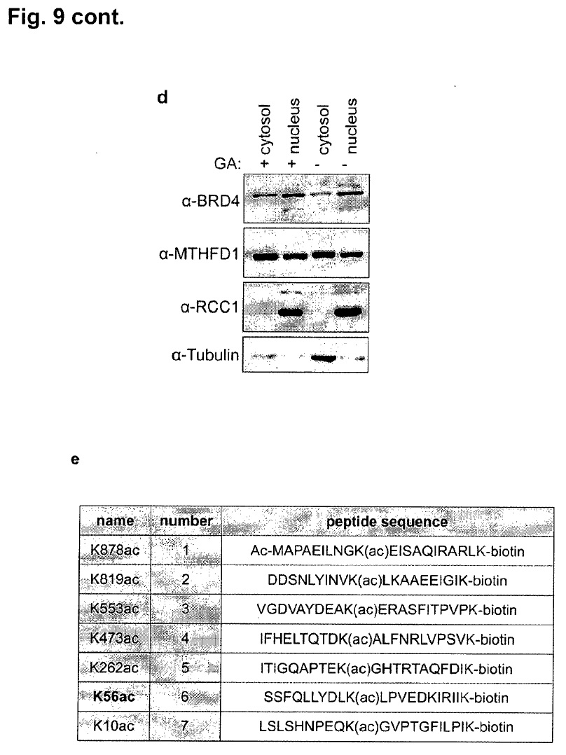

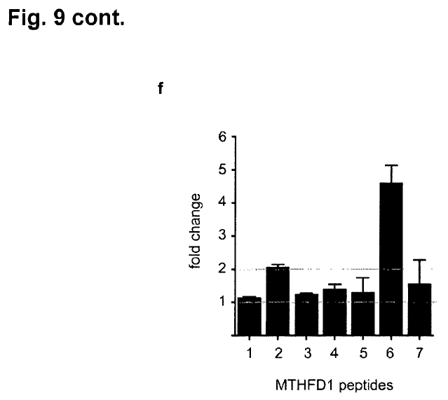

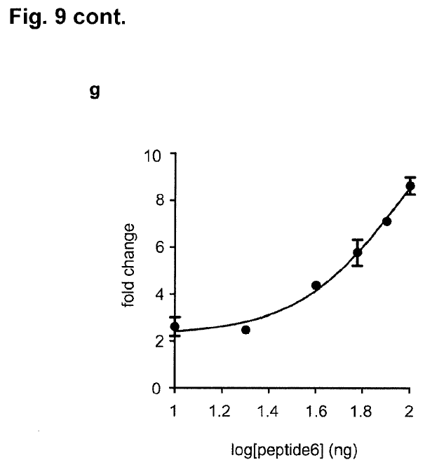

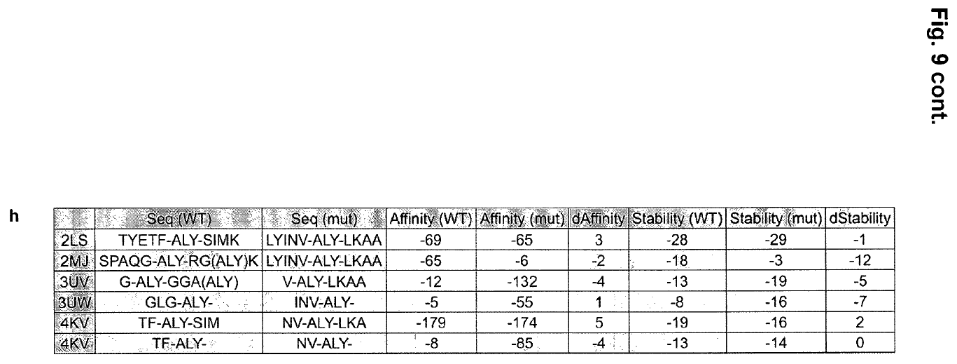

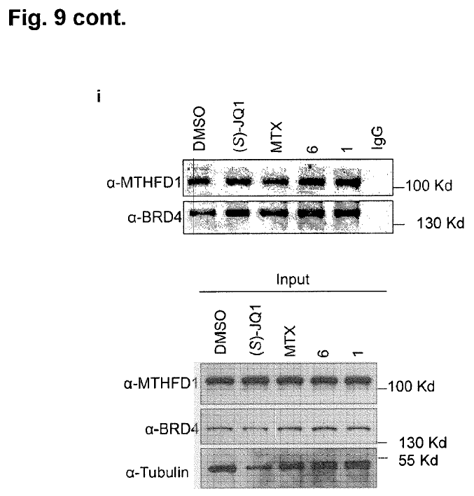

Thus, it is most preferred that the amount of nuclear MTHFD1 is determined. Immunofluorescence staining and immunohistochemistry are suitable methods for staining the protein with specific antibodies, and determination of the levels of the fluorescence signal in the nucleus (e.g., by co-staining with a DNA dye like DAPI, Hoechst 33258 or Hoechst 33342). Alternatively, nuclei can be isolated from tumor biopsies similarly to the isolation from cell lines described in FIG. 9. From a nuclear lysate, MTHFD1 levels can be determined using technologies like, for example, any one of: western blotting, ELISA and other immunological detection methods; enzymatic methods based on detecting the substrates and products of the MTHFD1 catalytic steps; and proteomic methods.

MTHFD1 is a C-1-tetrahydrofolate synthase that catalyzes three enzymatic reactions in folate metabolism, resulting in the interconversion of tetrahydrofolate (THF), 10-formyltetrahydrofolate (10-CHO-THF), 5,10-methenyltetrahydrofolate (5,10-CH=THF) and 5,10-methylenetetrahydrofolate (5,10-CH₂-THF). It has been observed by the inventors that knock-down of either MTHFD1 or BRD4 resulted in lower levels of 5,10-CH₂-THF. The nuclear levels of all folate metabolites can be determined following the isolation of nuclei, lysis, precipitation of proteins and analysis with methods including, e.g., HPLC-MS/MS and antibody-based methods like ELISA.

The present invention furthermore relates to a BRD4 inhibitor for use in the treatment of cancer in a subject, wherein the subject has been identified in any of the above-described methods as being susceptible or responsive to the treatment with a BRD4 inhibitor.

Moreover, the invention relates to the use of (i) a pair of primers for (i.e., binding to) a transcript of the gene MTHFD1, (ii) a nucleic acid probe to (i.e., binding to) a transcript of the gene MTHFD1, (iii) a microarray comprising a nucleic acid probe to (i.e., binding to) the transcript of the gene MTHFD1, or (iv) an antibody against (i.e., binding to) the protein MTHFD1, in a method (particularly an in vitro method) of assessing the susceptibility or responsiveness of a subject to the treatment with a BRD4 inhibitor, wherein the subject has been diagnosed as suffering from cancer or is suspected of suffering from cancer (e.g., any of the corresponding methods as described herein above).

The primers can be designed using methods known in the art (as also described, e.g., in Green et al., 2012) so as to allow the specific amplification/quantification of the transcript of the gene MTHFD1. Furthermore, the primers are preferably DNA primers.

The above-mentioned transcript is preferably an mRNA of the gene MTHFD1 or a cDNA synthesized from the mRNA of the gene MTHFD1. The nucleic acid probe comprises or consists of a nucleic acid capable of hybridizing with the transcript. The nucleic acid probe is preferably a single-stranded DNA probe or a single-stranded RNA probe, more preferably a single-stranded DNA probe. It is furthermore preferred that the nucleic acid probe (which may be, e.g., a single-stranded DNA or a single-stranded RNA, and is preferably a single-stranded DNA) is an oligonucleotide probe having, e.g., 10 to 80 nucleotides, preferably 15 to 60 nucleotides, more preferably 20 to 35 nucleotides, and even more preferably about 25 nucleotides. Such nucleic acid probes can be designed using methods known in the art (as also described, e.g., in Green et al., 2012) so as to allow the specific detection and quantification of the transcript of the corresponding gene.

The above-mentioned antibody against the protein MTHFD1 binds specifically to the protein MTHFD1 and may be, e.g., a polyclonal antibody or a monoclonal antibody. Preferably, the antibody is a monoclonal antibody. The antibody may further be a full/intact immunoglobulin molecule or a fragment/part thereof (such as, e.g., a separated light or heavy chain, an Fab fragment, an Fab/c fragment, an Fv fragment, an Fab′ fragment, or an F(ab′)₂ fragment), provided that the fragment/part substantially retains the binding specificity of the corresponding full immunoglobulin molecule. The antibody may also be a modified and/or altered antibody, such as a chimeric or humanized antibody, a bifunctional or trifunctional antibody, or an antibody construct (such as a single-chain variable fragment (scFv) or an antibody-fusion protein). The antibody can be prepared using methods known in the art, as also described, e.g., in Harlow, E. et al., Using Antibodies: A Laboratory Manual, Cold Spring Harbor Laboratory Press, 1998, ISBN: 978-0879695446. For example, monoclonal antibodies can be prepared by methods such as the hybridoma technique (see, e.g., Köhler G, et al. Nature. 1975; 256(5517):495-7), the trioma technique, the human B-cell hybridoma technique (see, e.g., Kozbor D, et al. Immunol Today. 1983; 4(3):72-9) or the EBV-hybridoma technique (see, e.g., Cole S P C, et al. Monoclonal Antibodies and Cancer Therapy. 1985; 27:77-96).

Thus, as described above, the present invention provides in particular:

(i) A BRD4 inhibitor for use in a method of treating cancer in a subject that has been diagnosed as suffering from cancer or is suspected of suffering from cancer, the method comprising:
determining the level of nuclear folate and/or the level of expression of MTHFD1 in a sample obtained from the subject;
determining whether or not the subject is susceptible or responsive to the treatment with a BRD4 inhibitor, wherein a smaller level of nuclear folate and/or a smaller expression level of MTHFD1 in the sample from the subject is/are indicative of the subject being more susceptible or more responsive to the treatment with a BRD4 inhibitor; and
administering a BRD4 inhibitor to the subject if the subject has been identified as being susceptible or responsive to the treatment with a BRD4 inhibitor.

(ii) A BRD4 inhibitor for use in a method of treating cancer in a subject that has been diagnosed as suffering from cancer or is suspected of suffering from cancer, the method comprising:
determining the level of nuclear folate in a sample obtained from the subject;
determining whether or not the subject is susceptible or responsive to the treatment with a BRD4 inhibitor, wherein a smaller level of nuclear folate in the sample from the subject is indicative of the subject being more susceptible or more responsive to the treatment with a BRD4 inhibitor; and
administering a BRD4 inhibitor to the subject if the subject has been identified as being susceptible or responsive to the treatment with a BRD4 inhibitor.

(iii) A BRD4 inhibitor for use in a method of treating cancer in a subject that has been diagnosed as suffering from cancer or is suspected of suffering from cancer, the method comprising:
determining the level of expression of MTHFD1 in a sample obtained from the subject;
determining whether or not the subject is susceptible or responsive to the treatment with a BRD4 inhibitor, wherein a smaller expression level of MTHFD1 in the sample from the subject is indicative of the subject being more susceptible or more responsive to the treatment with a BRD4 inhibitor; and
administering a BRD4 inhibitor to the subject if the subject has been identified as being susceptible or responsive to the treatment with a BRD4 inhibitor.

(iv) A BRD4 inhibitor for use in a method of treating cancer in a subject that has been diagnosed as suffering from cancer or is suspected of suffering from cancer, the method comprising:
determining the level of nuclear MTHFD1 protein in a sample obtained from the subject;
determining whether or not the subject is susceptible or responsive to the treatment with a BRD4 inhibitor, wherein a smaller level of nuclear MTHFD1 protein in the sample from the subject is indicative of the subject being more susceptible or more responsive to the treatment with a BRD4 inhibitor; and
administering a BRD4 inhibitor to the subject if the subject has been identified as being susceptible or responsive to the treatment with a BRD4 inhibitor.

The compounds to be used in accordance with the invention may be administered as compounds per se or may be formulated as medicaments or pharmaceutical compositions. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cycfodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds to be used in accordance with the invention or the above described pharmaceutical compositions comprising such compounds may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e,g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides (see, e.g., U.S. Pat. No. 3,773,919), copolymers of L-glutamic acid and gamma-ethyl-L-glutamate (Sidman, U. et al., Biopolymers 22:547-556 (1983)), poly(2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP133988). Sustained-release pharmaceutical compositions also include liposomally entrapped compounds. Liposomes containing a compound of the present invention can be prepared by methods known in the art, such as, e.g., the methods described in any one of: DE3218121; Epstein et al., Proc. Natl. Acad. Sci. (USA) 82:3688-3692 (1985); Hwang et al., Proc. Natl. Acad. Sci, (USA) 77:4030-4034 (1980); EP0052322; EP0036676; EP088046; EP0143949; EP0142641; JP 83-118008; U.S. Pat. No. 4,485,045; U.S. Pat. No. 4,544,545; and EP0102324.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds to be used in accordance with the invention for pulmonary administration, particularly inhalation. Such dry powders may be prepared by spray drying under conditions which result in a substantially amorphous glassy or a substantially crystalline bioactive powder. Accordingly, dry powders of the compounds to be used in the present invention can be made according to the emulsification/spray drying process disclosed in WO 99/16419 or WO 01/85136. Spray drying of solution formulations of the respective compounds can be carried out, e.g., as described generally in the “Spray Drying Handbook”, 5th ed., K. Masters, John Wiley & Sons, Inc., NY (1991), in WO 97/41833, or in WO 03/053411.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compounds or pharmaceutical compositions are to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Particularly preferred routes of administration are oral administration or parenteral administration.

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

The combination of a BRD4 inhibitor with an antifolate (or with an MTHFD1 inhibitor) according to the present invention can also be used in combination with other therapeutic agents, including in particular other anticancer agents, for the treatment or prevention of cancer. When the above-mentioned drug combination according to the present invention is used in combination with a further therapeutic agent active against the same disease, the dose of each compound may differ from that when the compound is used alone. The combination of the drug combination of the present invention with a further therapeutic agent may comprise the administration of the further therapeutic agent simultaneously/concomitantly or sequentially/separately with the compounds of the drug combination according to the invention.

Preferably, the further therapeutic agent to be administered in combination with the compounds of the drug combination of the present invention is an anticancer drug. The anticancer drug may be selected from: a tumor angiogenesis inhibitor (e.g., a protease inhibitor, an epidermal growth factor receptor kinase inhibitor, or a vascular endothelial growth factor receptor kinase inhibitor); a cytotoxic drug (e.g., an antimetabolite, such as purine and pyrimidine analog antimetabolites); an antimitotic agent (e.g., a microtubule stabilizing drug or an antimitotic alkaloid); a platinum coordination complex; an anti-tumor antibiotic; an alkylating agent (e.g., a nitrogen mustard or a nitrosourea); an endocrine agent (e.g., an adrenocorticosteroid, an androgen, an anti-androgen, an estrogen, an anti-estrogen, an aromatase inhibitor, a gonadotropin-releasing hormone agonist, or a somatostatin analog); or a compound that targets an enzyme or receptor that is overexpressed and/or otherwise involved in a specific metabolic pathway that is misregulated in the tumor cell (e.g., ATP and GTP phosphodiesterase inhibitors, histone deacetylase inhibitors, protein kinase inhibitors (such as serine, threonine and tyrosine kinase inhibitors, e.g., Abelson protein tyrosine kinase inhibitors) and the various growth factors, their receptors and corresponding kinase inhibitors (such as epidermal growth factor receptor kinase inhibitors, vascular endothelial growth factor receptor kinase inhibitors, fibroblast growth factor inhibitors, insulin-like growth factor receptor inhibitors and platelet-derived growth factor receptor kinase inhibitors)); methionine, aminopeptidase inhibitors, proteasome inhibitors, cyclooxygenase inhibitors (e.g., cyclooxygenase-1 or cyclooxygenase-2 inhibitors), topoisomerase inhibitors (e.g., topoisomerase I inhibitors or topoisomerase II inhibitors), poly ADP ribose polymerase inhibitors (PARP inhibitors), and epidermal growth factor receptor (EGFR) inhibitors/antagonists.

An alkylating agent which can be used as an anticancer drug in combination with the compounds of the drug combination of the present invention may be, for example, a nitrogen mustard (such as cyclophosphamide, mechlorethamine (chlormethine), uramustine, melphalan, chlorambucil, ifosfamide, bendamustine, or trofosfamide), a nitrosourea (such as carmustine, streptozocin, fotemustine, lomustine, nimustine, prednimustine, ranimustine, or semustine), an alkyl sulfonate (such as busulfan, mannosulfan, or treosulfan), an aziridine (such as hexamethylmelamine (altretamine), triethylenemelamine, ThioTEPA (N,N′N′-triethylenethiophosphoramide), carboquone, or triaziquone), a hydrazine (such as procarbazine), a triazene (such as dacarbazine), or an imidazotetrazine (such as temozolomide).

A platinum coordination complex which can be used as an anticancer drug in combination with the compounds of the drug combination of the present invention may be, for example, cisplatin, carboplatin, nedaplatin, oxaliplatin, satraplatin, or triplatin tetranitrate.

A cytotoxic drug which can be used as an anticancer drug in combination with the compounds of the drug combination of the present invention may be, for example, an antimetabolite, including folic acid analogue antimetabolites (such as aminopterin, methotrexate, pemetrexed, or raltitrexed), purine analogue antimetabolites (such as cladribine, clofarabine, fludarabine, 6-mercaptopurine (including its prodrug form azathioprine), pentostatin, or 6-thioguanine), and pyrimidine analogue antimetabolites (such as cytarabine, decitabine, 5-fluorouracil (including its prodrug forms capecitabine and tegafur), floxuridine, gemcitabine, enocitabine, or sapacitabine).

An antimitotic agent which can be used as an anticancer drug in combination with the compounds of the drug combination of the present invention may be, for example, a taxane (such as docetaxel, larotaxel, ortataxel, paclitaxel/taxol, or tesetaxel), a Vinca alkaloid (such as vinblastine, vincristine, vinflunine, vindesine, or vinorelbine), an epothilone (such as epothilone A, epothilone B, epothilone C, epothilone D, epothilone E, or epothilone F) or an epothilone B analogue (such as ixabepilone/azaepothilone B).

An anti-tumor antibiotic which can be used as an anticancer drug in combination with the compounds of the drug combination of the present invention may be, for example, an anthracycline (such as aclarubicin, daunorubicin, doxorubicin, epirubicin, idarubicin, amrubicin, pirarubicin, valrubicin, or zorubicin), an anthracenedione (such as mitoxantrone, or pixantrone) or an anti-tumor antibiotic isolated from Streptomyces (such as actinomycin (including actinomycin D), bleomycin, mitomycin (including mitomycin C), or plicamycin),

A tyrosine kinase inhibitor which can be used as an anticancer drug in combination with the compounds of the drug combination of the present invention may be, for example, axitinib, bosutinib, cediranib, dasatinib, erlotinib, gefitinib, imatinib, lapatinib, lestaurtinib, nilotinib, semaxanib, sorafenib, sunitinib, or vandetanib.

A topoisomerase-inhibitor which can be used as an anticancer drug in combination with the compounds of the drug combination of the present invention may be, for example, a topoisomerase I inhibitor (such as irinotecan, topotecan, camptothecin, belotecan, rubitecan, or lamellarin D) or a topoisomerase II inhibitor (such as amsacrine, etoposide, etoposide phosphate, teniposide, or doxorubicin).

A PARP inhibitor which can be used as an anticancer drug in combination with the compounds of the drug combination of the present invention may be, for example, BMN-673, olaparib, rucaparib, veliparib, CEP 9722, MK 4827, BGB-290, or 3-aminobenzamide.

An EGFR inhibitor/antagonist which can be used as an anticancer drug in combination with the compounds of the drug combination of the present invention may be, for example, gefitinib, erlotinib, lapatinib, afatinib, neratinib, ABT-414, dacomitinib, AV-412, PD 153035, vandetanib, PKI-166, pelitinib, canertinib, icotinib, poziotinib, BMS-690514, CUDC-101, AP26113, XL647, cetuximab, panitumumab, zalutumumab, nimotuzumab, or matuzumab.

Further anticancer drugs may also be used in combination with the compounds of the drug combination of the present invention. The anticancer drugs may comprise biological or chemical molecules, like TNF-related apoptosis-inducing ligand (TRAIL), tamoxifen, amsacrine, bexarotene, estramustine, irofulven, trabectedin, cetuximab, panitumumab, tositumomab, alemtuzumab, bevacizumab, edrecolomab, gemtuzumab, alvocidib, seliciclib, aminolevulinic acid, methyl aminolevulinate, efaproxiral, porfimer sodium, talaporfin, temoporfin, verteporfin, alitretinoin, tretinoin, anagrelide, arsenic trioxide, atrasentan, bortezomib, carmofur, celecoxib, demecolcine, elesclomol, elsamitrucin, etoglucid, lonidamine, lucanthone, masoprocol, mitobronitol, mitoguazone, mitotane, oblimersen, omacetaxine, sitimagene, ceradenovec, tegafur, testolactone, tiazofurine, tipifarnib, vorinostat, or iniparib.

Also biological drugs, like antibodies, antibody fragments, antibody constructs (for example, single-chain constructs), and/or modified antibodies (like CDR-grafted antibodies, humanized antibodies, “full humanized” antibodies, etc.) directed against cancer or tumor markers/factors/cytokines involved in proliferative diseases can be employed in co-therapy approaches with the compounds of the drug combination of the present invention. Examples of such biological molecules are anti-HER2 antibodies (e.g. trastuzumab, Herceptin®), anti-CD20 antibodies (e.g. Rituximab, Rituxan®, MabThera®, Reditux®), anti-CD19/CD3 constructs (see, e.g., EP1071752) and anti-TNF antibodies (see, e.g., Taylor P C. Antibody therapy for rheumatoid arthritis. Curr Opin Pharmacol. 2003. 3(3):323-328). Further antibodies, antibody fragments, antibody constructs and/or modified antibodies to be used in co-therapy approaches with the compounds of the drug combination of the invention can be found, e.g., in: Taylor P C. Curr Opin Pharmacol, 2003. 3(3):323-328; or Roxana A. Maedica. 2006. 1(1):63-65.

An anticancer drug which can be used in combination with the compounds of the drug combination of the present invention may, in particular, be an immunooncology therapeutic (such as an antibody (e.g., a monoclonal antibody or a polyclonal antibody), an antibody fragment, an antibody construct (e.g., a single-chain construct), or a modified antibody (e.g., a CDR-grafted antibody, a humanized antibody, or a “full humanized” antibody) targeting any one of CTLA-4, PD-1/PD-L1, TIM3, LAG3, OX4, CSF1R, IDO, or CD40. Such immunooncology therapeutics include, e.g., an anti-CTLA-4 antibody (particularly an antagonistic or pathway-blocking anti-CTLA-4 antibody; e.g., ipilimumab or tremelimumab), an anti-PD-1 antibody (particularly an antagonistic or pathway-blocking anti-PD-1 antibody; e.g., nivolumab (BMS-936558), pembrolizumab (MK-3475), pidilizumab (CT-011), AMP-224, or APE02058), an anti-PD-L1 antibody (particularly a pathway-blocking anti-PD-L1 antibody; e.g., BMS-936559, MEDI4736, MPDL3280A (RG7446), MDX-1105, or MEDI6469), an anti-TIM3 antibody (particularly a pathway-blocking anti-TIM3 antibody), an anti-LAG3 antibody (particularly an antagonistic or pathway-blocking anti-LAG3 antibody; e.g., BMS-986016, IMP701, or IMP731), an anti-OX4 antibody (particularly an agonistic anti-OX4 antibody; e.g., MEDI0562), an anti-CSF1R antibody (particularly a pathway-blocking anti-CSF1R antibody; e.g., IMC-CS4 or RG7155), an anti-IDO antibody (particularly a pathway-blocking anti-IDO antibody), or an anti-CD40 antibody (particularly an agonistic anti-CD40 antibody; e.g., CP-870,893 or Chi Lob 7/4). Further immunooncology therapeutics are known in the art and are described, e.g., in: Kyi C et al., FEBS Lett, 2014, 588(2):368-76; Intlekofer A M et al., J Leukoc Biol, 2013, 94(1):25-39; Callahan M K et al., J Leukoc Biol, 2013, 94(1):41-53; Ngiow S F et al., Cancer Res, 2011, 71(21):6567-71; and Blattman J N et al., Science, 2004, 305(5681):200-5.

The combinations with further anticancer drugs referred to above may conveniently be presented for use in the form of a pharmaceutical formulation. The individual components of such combinations may be administered either sequentially or simultaneously/concomitantly in separate or combined pharmaceutical formulations by any convenient route. When administration is sequential, either the compounds of the drug combination of the present invention or the further therapeutic agent may be administered first. When administration is simultaneous, the combination may be administered either in the same pharmaceutical composition or in different pharmaceutical compositions. When combined in the same formulation, it will be appreciated that the different compounds must be stable and compatible with each other and the other components of the formulation. When formulated separately, they may be provided in any convenient formulation.

The compounds of the drug combination of the present invention can also be administered in combination with physical therapy, such as radiotherapy. Radiotherapy may commence before, after, or simultaneously with administration of the compounds of the drug combination of the present invention. For example, radiotherapy may commence 1-10 minutes, 1-10 hours or 24-72 hours after administration of the corresponding compounds. Yet, these time frames are not to be construed as limiting. The subject is exposed to radiation, preferably gamma radiation, whereby the radiation may be provided in a single dose or in multiple doses that are administered over several hours, days and/or weeks. Gamma radiation may be delivered according to standard radiotherapeutic protocols using standard dosages and regimens.

The present invention thus relates to a combination of a BRD4 inhibitor with an antifolate (or with an MTHFD1 inhibitor), as described herein above, for use in treating or preventing cancer, wherein the compounds of this drug combination (i.e., the BRD4 inhibitor and the antifolate or the MTHFD1 inhibitor, or a pharmaceutical composition comprising these agents) are to be administered in combination with a further anticancer drug and/or in combination with radiotherapy.

The subject or patient to be treated in accordance with the present invention may be an animal (e.g., a non-human animal), a vertebrate animal, a mammal, a rodent (e.g., a guinea pig, a hamster, a rat, or a mouse), a canine (e.g., a dog), a feline (e.g., a cat), a porcine (e.g., a pig), an equine (e.g., a horse), a primate or a simian (e.g., a monkey or an ape, such as a marmoset, a baboon, a gorilla, a chimpanzee, an orangutan, or a gibbon), or a human. In accordance with the present invention, it is envisaged that animals are to be treated which are economically, agronomically or scientifically important. Scientifically important organisms include, but are not limited to, mice, rats, and rabbits, Lower organisms such as, e.g., fruit flies like Drosophila melagonaster and nematodes like Caenorhabditis elegans may also be used in scientific approaches. Non-limiting examples of agronomically important animals are sheep, cattle and pigs, while, for example, cats and dogs may be considered as economically important animals. Preferably, the subject/patient is a mammal. More preferably, the subject/patient is a human or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient is a human.

The term “treatment” of a disorder or disease as used herein (e.g., “treatment” of cancer) is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter aha, comprise curative treatment (preferably leading to a complete response and eventually to healing of the disorder or disease) and palliative treatment (including symptomatic relief).

The term “prevention” of a disorder or disease as used herein (e.g., “prevention” of cancer) is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

As used herein, unless explicitly indicated otherwise or contradicted by context, the terms “a”, “an” and “the” are used interchangeably with “one or more” and “at least one”. Thus, for example, a composition comprising “a” BRD4 inhibitor can be interpreted as referring to a composition comprising “one or more” BRD4 inhibitors.

As used herein, the term “about” preferably refers to ±10% of the indicated numerical value, more preferably to ±5% of the indicated numerical value, and in particular to the exact numerical value indicated. For example, the expression “about 100” preferably refers to 100±10%, more preferably to 100±5%, and even more preferably to the specific value of 100.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e, no other components than B and C are comprised in A).

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, if a component of a composition is indicated to be “optional”, the invention specifically relates to both possibilities, i.e., that the corresponding component is present (contained in the composition) or that the corresponding component is absent from the composition.

It is to be understood that the present invention specifically relates to each and every combination of features and embodiments described herein, including any combination of general and/or preferred features/embodiments.

In this specification, a number of documents including patent documents and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The invention is also described by the following illustrative figures. The appended figures show:

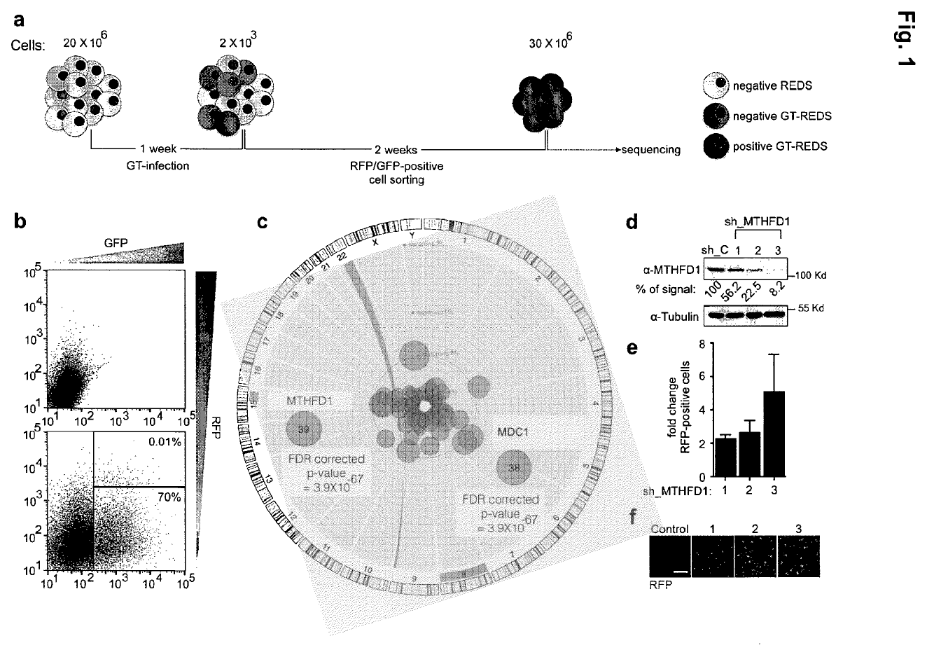

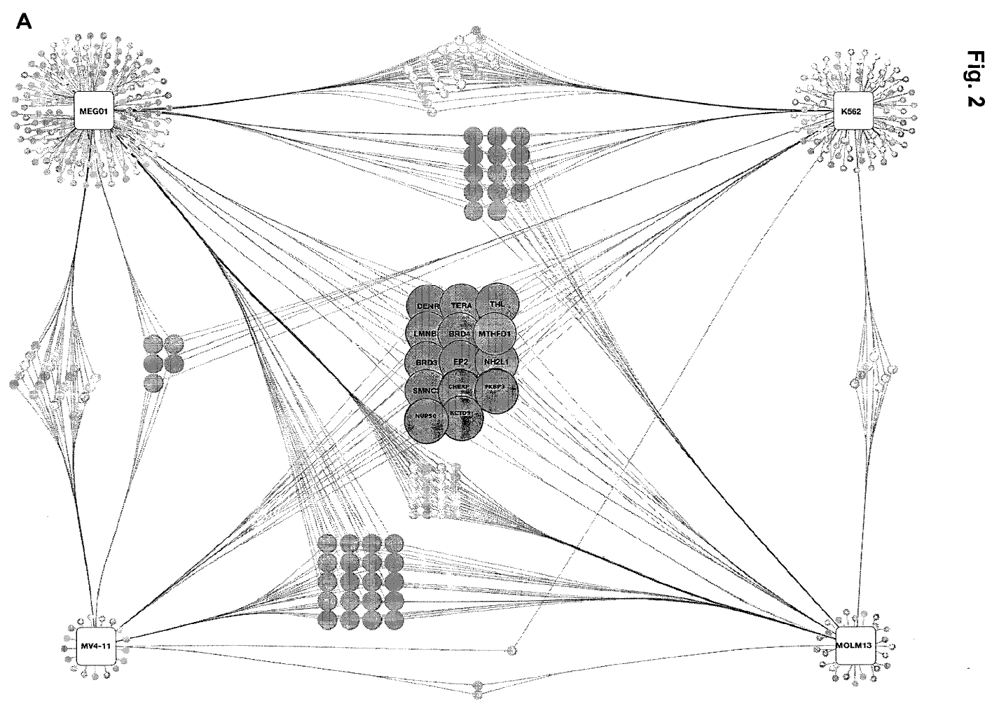

FIG. 1: A gene-trap based genetic screen identifies MTHFD1 as BRD4 partner. (A) Schematic overview of the gene-trap based genetic screen experimental approach. Briefly, REDS1 cells were infected with a gene-trap virus encoding for the GFP reporter gene. Gene-trapped cells could be recognised by GFP fluorescence. One week after infection, cells expressing GFP and RFP expression were FACS-sorted, amplified (during 2 additional weeks) and processed for sequencing. (B) Representative panels of the applied FACS-sorting strategy. The upper panel is non-infected REDS1 cells. The lower panel is gene-trap infected REDS1 cells; three population can be distinguished: non-infected cells (black), infected and GFP positive cells (green: 70%) and infected double positive (GFP/RFP) cells (red: 0.01%). the last population was sorted and sequenced. Three biological replicates were done for each experimental condition. (C) Circus-plot illustrating the hits from the gene-trap screen. Bubble size and distance from the centre are respectively proportional to the number of independent inactivating gene-trap sequenced integrations (direct proportion) and the p value (calculated with the Fisher Test; inverse proportion). (D) Western Blot showing MTHFD1 protein levels after downregulation with the indicated shRNAs in REDS3; numbers below MTHFD1 blot indicate the percentage of remaining MTHFD1 protein. Tubulin was used as loading control. (E) Quantification of RFP positive cells from live-cell imaging pictures of REDS1 cells treated with MTHFD1 shRNA. Three biological replicates were done for each experimental condition (mean±STD). (F) Representative live-cell imaging pictures of MTHFD1 knock down in REDS1 cells. RFP signal is shown in white; scale bar is 100 μm.

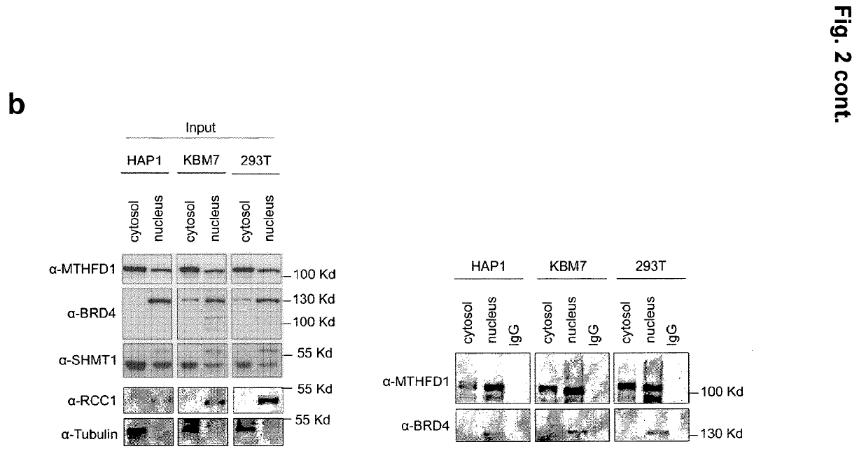

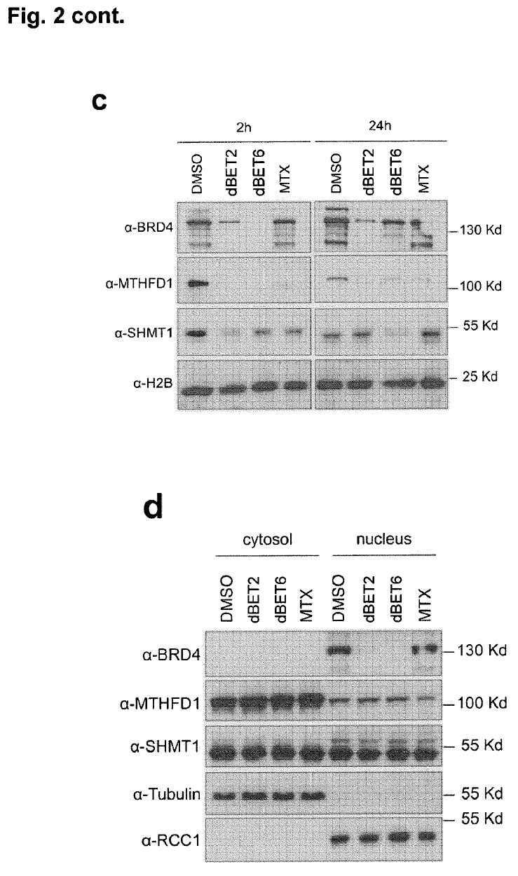

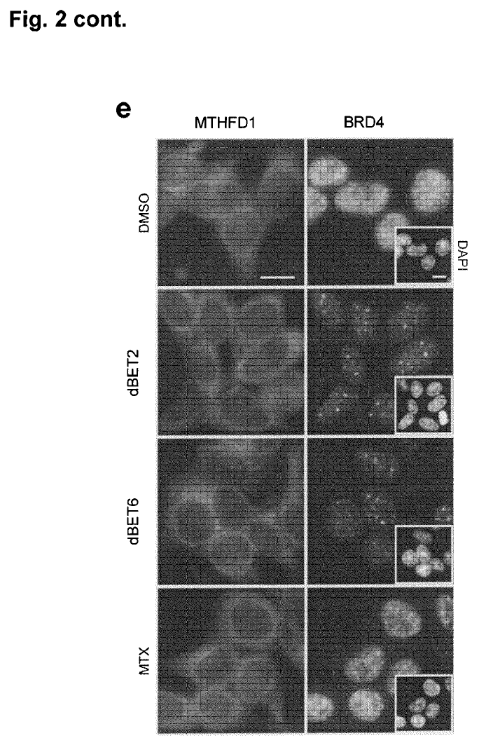

FIG. 2: BRD4 is essential for MTHFD1 recruitment on the chromatin. (A) Representation of BRD4 pull down performed in MEG01, K562, MV4-11 and MOLM-13 (with squares at the edges). Proteins are represented as circles: the dimension of the circle indicates the number of cell lines in which that protein has been found as BRD4 interactor. (B) Upper panel: Western Blot showing the level of the indicated proteins upon nuclear vs cytosol fractionation in HAP1, KBM7 and HEK293T (293T) cell lines. RCC1 was used as nuclear loading control while tubulin was used as cytosolic loading control. Lower panel: Western Blot showing MTHFD1 pull-down assay performed on whole cell lysate of the three cell line reported above. (C) Western Blot assay performed on chromatin associated protein samples extracted from HAP1 cells treated with the indicated compounds for 2 (dBET1: 0.5 μM; dBET6: 0.5 μM; MTX: 1 μM) or 24 hours (dBET1: 0.5 μM; dBET6: 0.05 μM; MTX: 1 μM). H2B was used as chromatin loading control. (D) Western Blot showing the level of the indicated proteins upon nuclear vs cytosol fractionation in HAP1 cells treated with the indicated compounds for 24 hours (dBET1: 0.5 μM; dBET6: 0.05 μM; MIX: 1 μM). RCC1 was used as nuclear loading control while tubulin was used as cytosolic loading control. (E) Immunofluorescence pictures of HELA cells treated with the indicated compounds and stained for BRD4, MTHFD1 and DAPI, as indicated in the figure (DAPI is shown in the small squares inside the BRD4 stained squares). Scale bar is 10 μm.

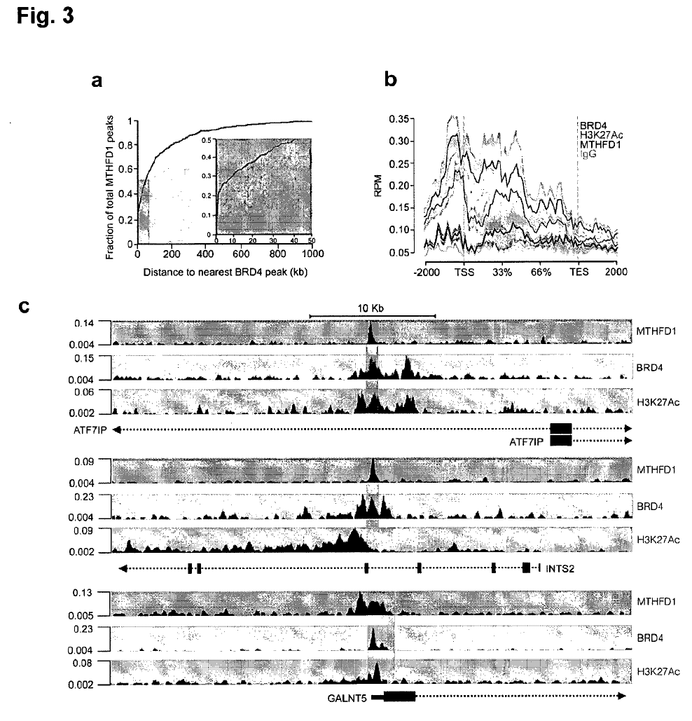

FIG. 3: MTHFD1 genome occupancy significantly overlaps with BRD4. (A) Graphic representation of the distance between BRD4 and MTHFD1 peaks; the small grey rectangle on the left (0.5 of the fraction of total MTHFD1 peaks/up to 50 kb distance from BRD4) is zoomed out in the smaller grey graph. (B) Representation of the genebody coverage of MTHFD1 (dark grey), H3K27Ac (grey), BRD4 (light grey) and IgG (very light grey) on MTHFD1 peaks, TSS is transcription start site, TES is transcription ending site. (C) Representation of three genomic loci occupied by MTHFD1 (dark grey), H3K27Ac (grey), BRD4 (light grey).

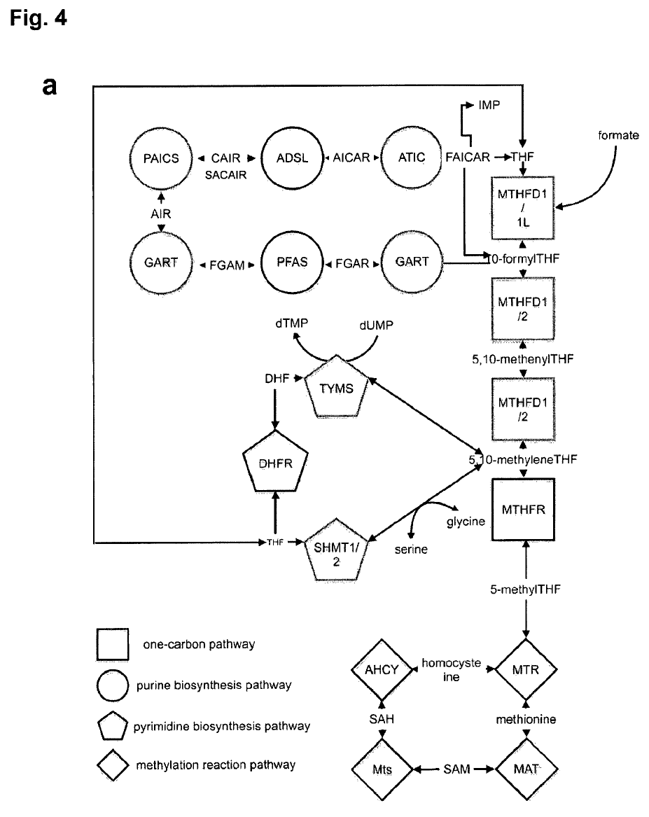

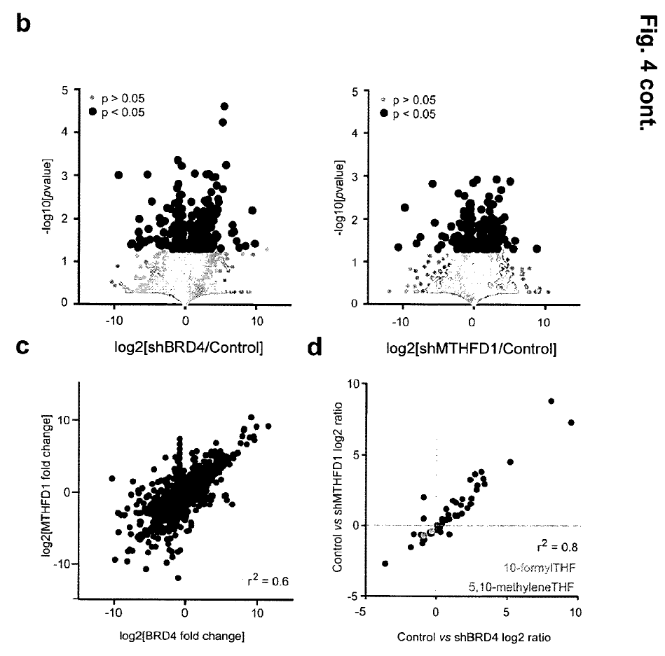

FIG. 4: MTHFD1 and BRD4 downregulation induce similar nuclear metabolomics changes. (A) Representation of the folate pathway. Enzyme names are reported inside the geometric shapes, while metabolites are written on the arrows. In white those enzymes that were found as in contact with chromatin. Chromatin associated proteins were extracted from HAP1 cells and analyzed by LC-MS. (B) Volcano plot representing metabolite fold change in BRD4 and MTHFD1 downregulated HAP1 cells. Dimension and color of the dots represent significantly (big and black) or not significantly (small and grey) altered nuclear metabolites. (C) Dot-plot showing the correlation (correlation coefficient 0.6) between changes induced by BRD4 or MTHFD1 downregulation on nuclear metabolites. (D) Dot-plot showing the correlation (correlation coefficient 0.8) between changes induced by BRD4 or MTHFD1 downregulation on nuclear folate metabolites.

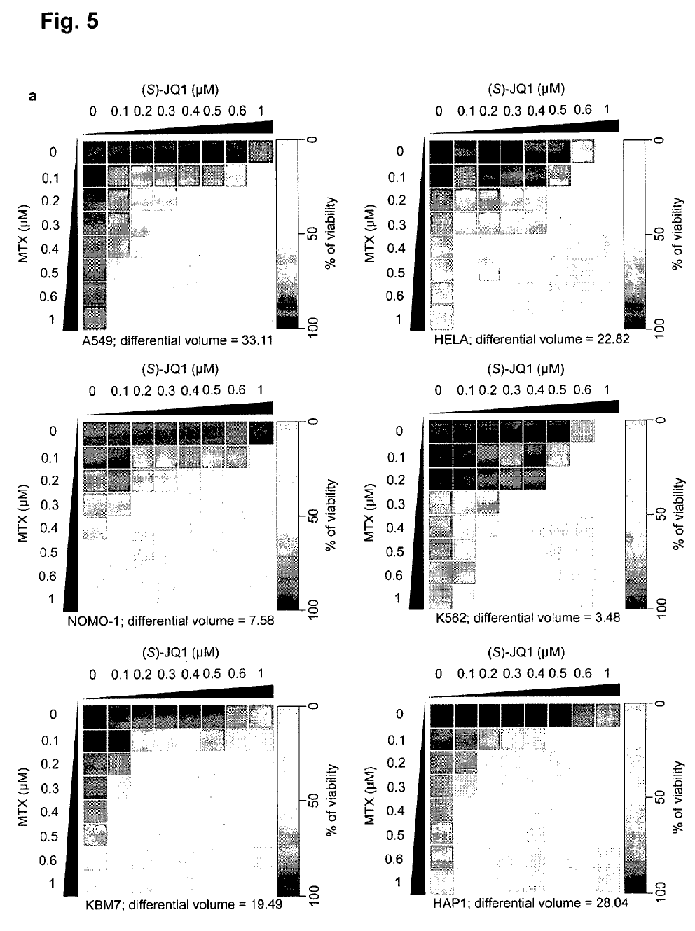

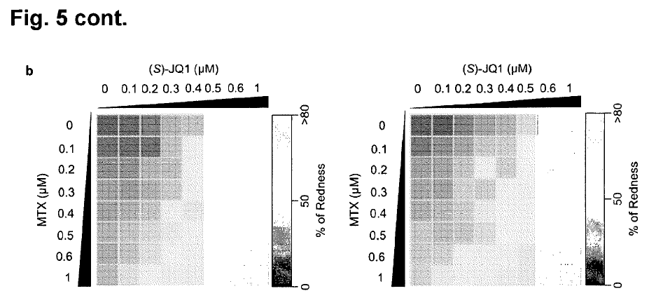

FIG. 5: (A) Matrix displaying cell viability reduction of H23 cells treated with the indicated concentrations of (S)-JQ1 and MTX alone or in combination (each point done in duplicate, an equal amount of DMSO was added as control). (B) Matrix displaying fold change of REDS1 RFP-positive cells treated with the indicated concentrations of (S)-JQ1 and MTX alone or in combination (each point done in duplicate, an equal amount of DMSO was added as control).

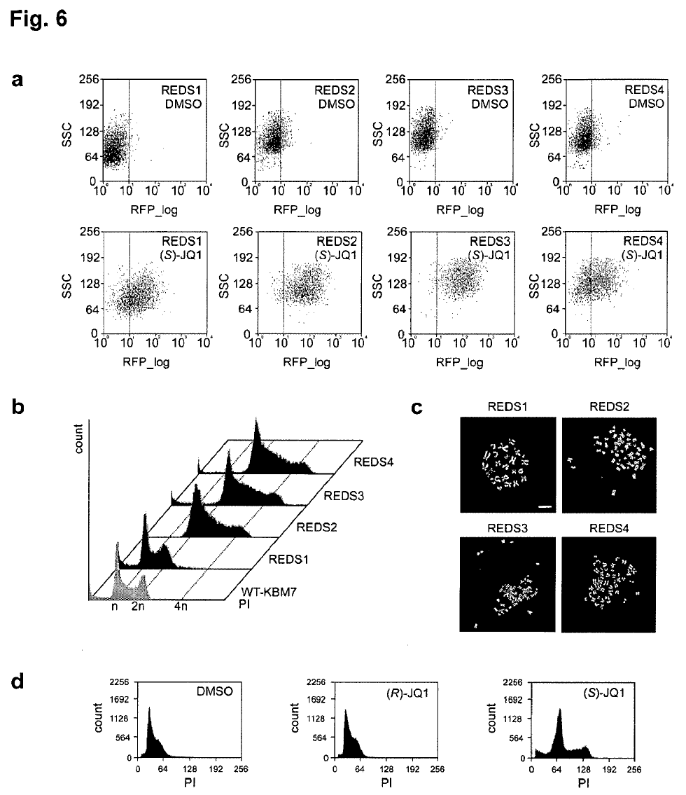

FIG. 6: (A) Representative FACS panels of REDS1, REDS2, REDS3 and REDS4 cells treated with 0.5 μM (S)-JQ1; an equal volume of DMSO was used as control. Three biological replicates were done for each experimental condition. (B) Representative cell cycle profiles evaluated by PI-staining and DNA content analysis by FACS. REDS1, REDS3 and REDS4 cells are compared to haploid WT-KBM7 (grey profile). Three biological replicates were done for each experimental condition. (C) Chromosomal spread preparation of metaphase nuclei stained with DAPI; scale bar 10 μm. Three biological replicates were done for each experimental condition. (D) Representative cell cycle profiles of cells treated overnight with (S)-JQ1 or (R)-JQ1 during one week, evaluated by PI-staining and DNA content analysis by FACS; an equal volume of DMSO was used as control. Three biological replicates were done for each experimental condition.

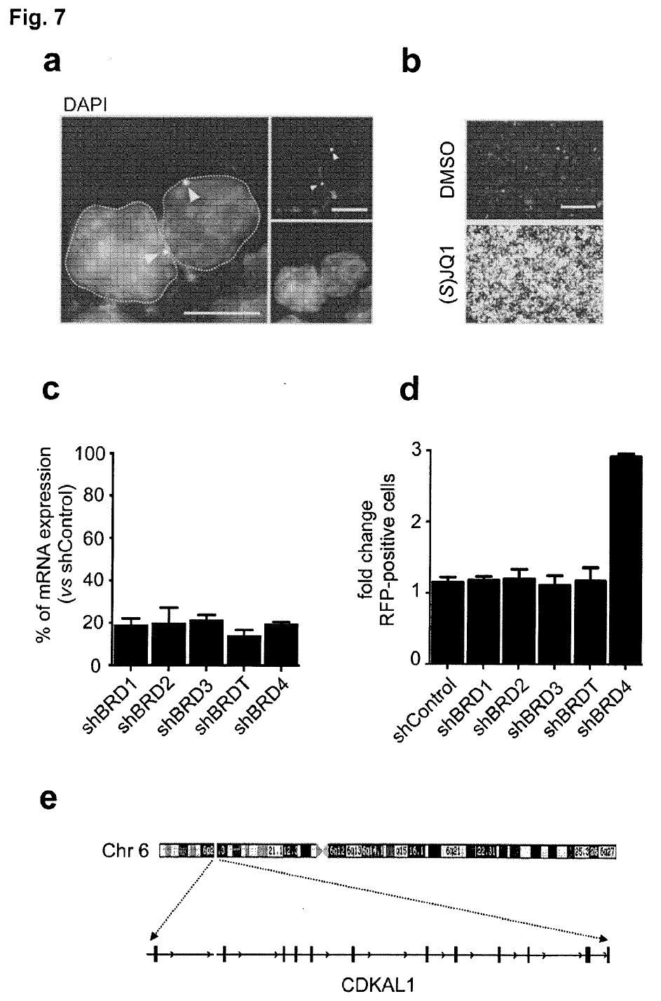

FIG. 7: (A) Representative pictures of the FISH assay done in REDS1 cells. RFP probe (white dots) stains the RFP insertion; DAPI (grey signal) stains the nucleus. Dashed lines mark nuclear perimeter. Scale bar is 10 μm. (B) Representative live cell imaging pictures of REDS1 treated with 1 μM (S)-JQ1 for 24 hours; an equal volume of DMSO was used as control. RFP expression is shown in red; scale bar is 100 μm. (C) BRD1, BRD2, BRD3, BRDT and BRD4 expression assessed by RT-PCR in BRD1, BRD2, BRD3, BRDT or BRD4 downregulated REDS1 cells; three biological replicates were done for each experimental condition (mean±STD). (D) Quantification of RFP positive cells from live imaging pictures of BRD1, BRD2, BRD3, BRDT or BRD4 downregulated REDS1 cells. Three biological replicates were done for each experimental condition (mean±STD). (E) Representation of the RFP locus. RFP is inserted in the antisense direction at 6 chromosome (chr6:20,520,542-20,588,419), in the first intron of CDKAL1 gene (sense direction).

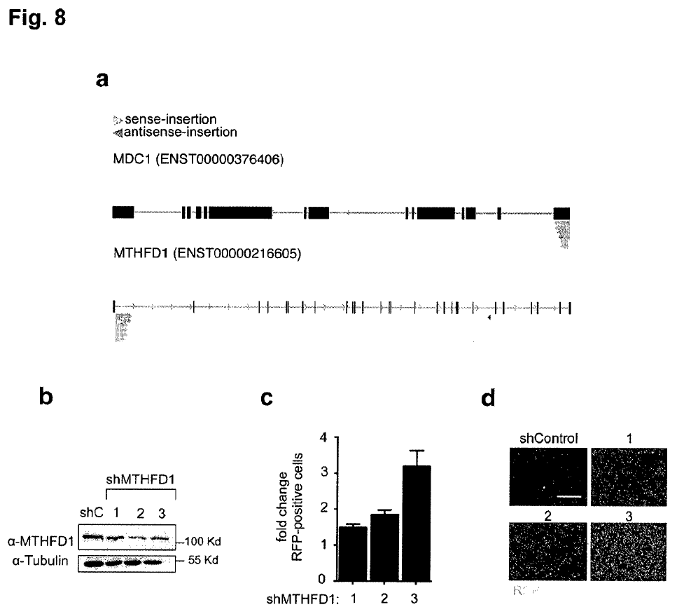

FIG. 8: (A) Representation of the gene-trap integration sites on MDC1 and MTHFD1 genes. Light grey arrows indicate sense insertions; grey arrows indicate antisense insertions. (B) Western Blot showing MTHFD1 protein levels after downregulation with the indicated shRNAs in REDS3. Tubulin was used as loading control. (C) Quantification of RFP positive cells from live-cell imaging pictures of REDS3 cells treated with MTHFD1 shRNA. Three biological replicates were done for each experimental condition (mean±STD). (D) Representative live-cell imaging pictures of MTHFD1 knock down REDS3 cells. RFP signal is shown in white; scale bar is 100 μm.

FIG. 9: (A) Western blot showing GFP pull-down using protein extracts from HEK293T overexpressing GFP-MTHFD1 or GFP alone. Tubulin was used as loading control. (B) Western Blot showing BRD4 pull-down in HELA cells. (C) Pipeline of the pull-down method used for the MS analysis. (D) Western Blot performed on the nuclear and cytosolic fractions of HAP1 cells treated with Ginkgolic Acid (GA) for 72 hours. RCC1 was used as nuclear loading control, while Tubulin was used as cytosolic loading control. (E) Table indicating the MTHFD1 acetylated peptides used in (F). (F) AlphaLISA assay performed with the indicated MTHFD1 acetylated peptides and GST-BRD4 (full length). The assay was done in duplicates (mean±STD). (G) AlphaLISA assay of MTHFD1-K56Ac peptide titration in combination with GST-BRD4 (full length). The assay was done in duplicates (mean±STD). (H) Binding of BRD4 bromodomains to acetylated MTHFD1(K56ac) peptide. Predicted affinity of the MTHFD1(K56ac) peptide (Affinity(mutant)) compared to the histone peptide co-crystallized with BRD4 (Affinity(WT)) calculated towards BRD4. More negative scores are indicative of higher affinity. Similarly, stabilities of the peptide in the bound configuration are calculated for the original histone (Stability(WT)) and the MTHFD1(K56ac) peptides. (I) Upper panel: MTHFD1 pull-down performed on HAP1 whole cell lysates treated with 50 μM (S)-JQ1, MTX, MTHFD1k56Ac (6) peptide or MTHFD1K878Ac peptide (1); equal amount of DMSO was used as control. Lower panel: Western Blot showing the level of the indicated proteins. HAP1 whole cell extracts were treated as before and Tubulin was used as loading control. (J) Docking of Methotrexate to the binding pocked of MTHFD1. Methotrexate is predicted to interact with Lysine 56 of MTHFD1 (left panel), and this interaction is lost when K56 is acetylated (right panel).

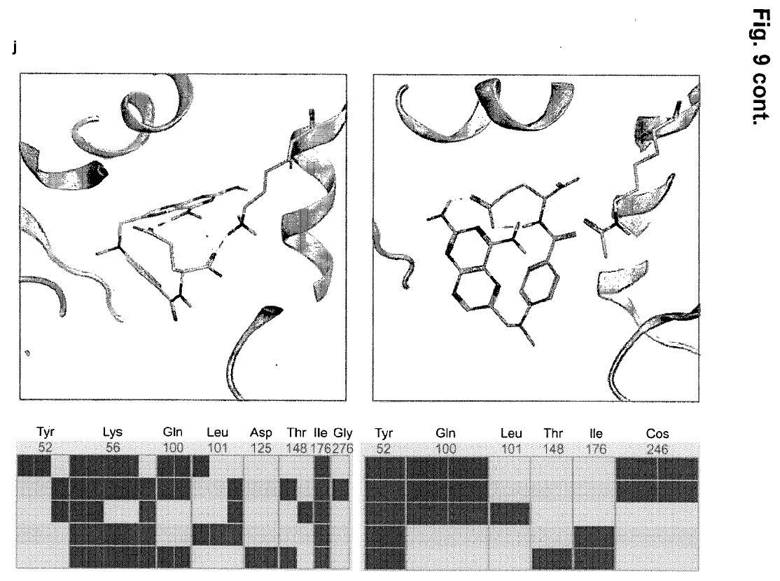

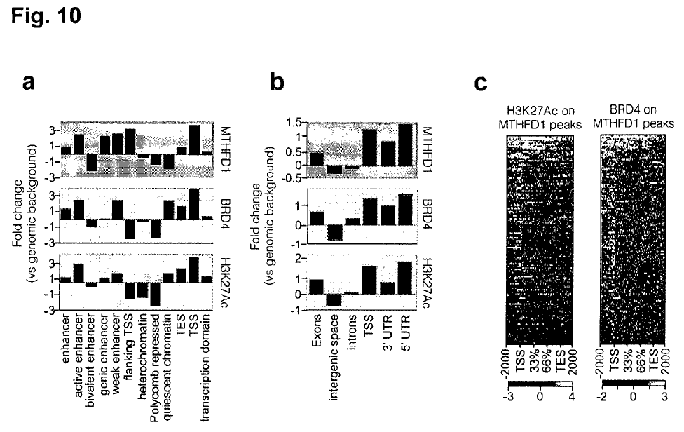

FIG. 10: MTHFD1 (dark grey), H3K27Ac (grey), BRD4 (light grey) occupancy at chromatin states (A) and genomic regions (B). TSS is transcription start site, TES is transcription ending site. (C) Heatmaps showing H3K27Ac genebody coverage of MTHFD1 peaks and BRD4 genebody coverage of MTHFD1 peaks.

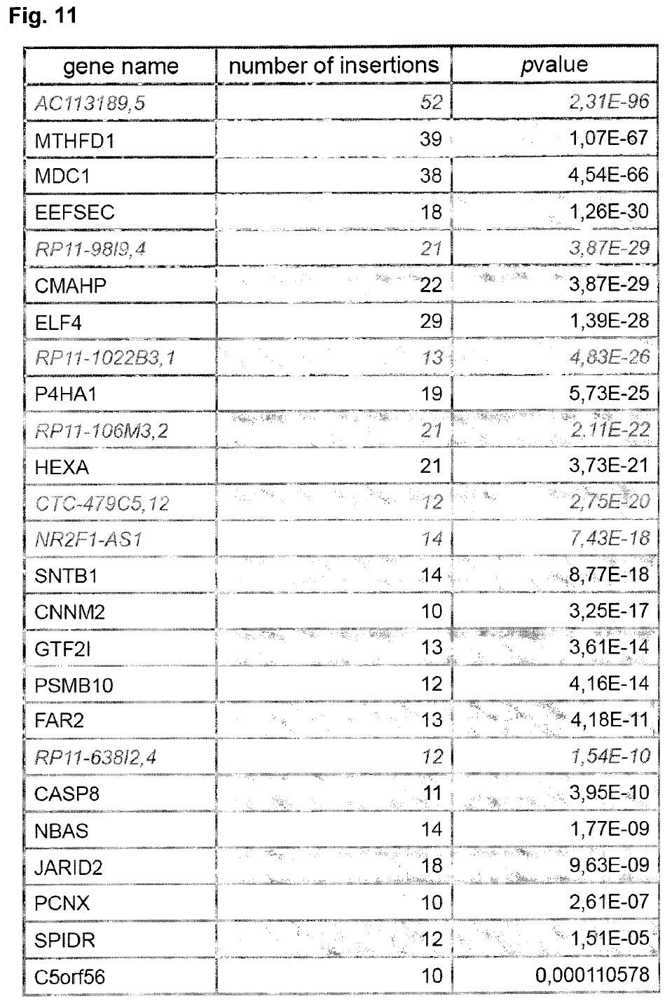

FIG. 11: List of the significant gene-trapped loci. The loci reported in the table were selected if showing more than 10 insertions in combination with a significant p value. In grey and italic are the identified non long coding RNA (not reported in the circus plot of FIG. 1C); in black and regular the identified coding genes. The values were calculated using the Fisher test.

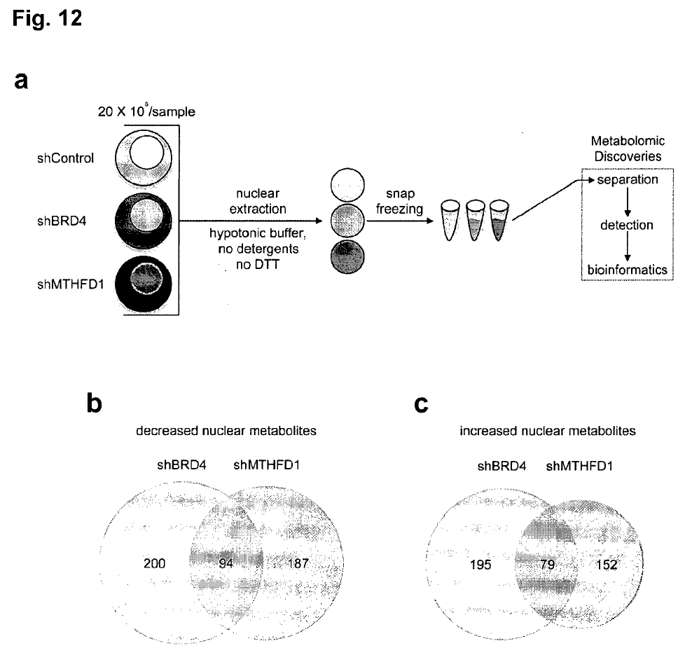

FIG. 12: (A) Illustration of the experimental workflow used for the nuclear metabolite sample preparation. Venn diagrams showing the overlap of significantly decreased (94) (B) or increased (79) (C) nuclear metabolites upon BRD4 or MTHFD1 downregulation.

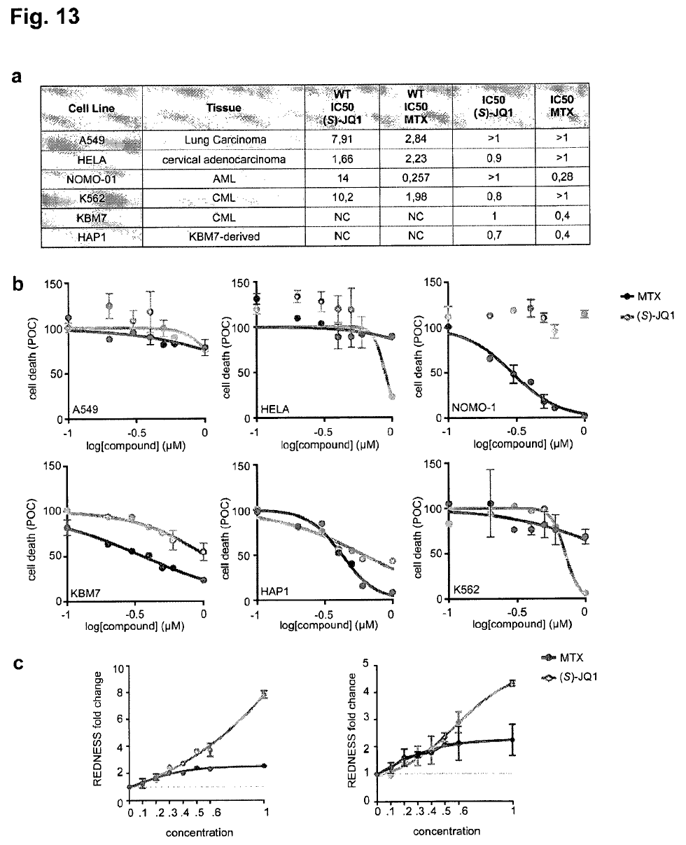

FIG. 13: (A) Table IC₅₀ values of (S)-JQ1 and MTX reported in the Welcome Trust-Genomics of Drug Sensitivity in Cancer database (WT; http://www.cancerrxgene.org) or produced in house. (B) (S)-JQ1 (grey) and MTX (dark grey) IC₅₀ determination in the indicated cell lines. (C) Redness fold change upon (S)-JQ1 (grey) or MTX (dark grey) titration in REDS1 clone.

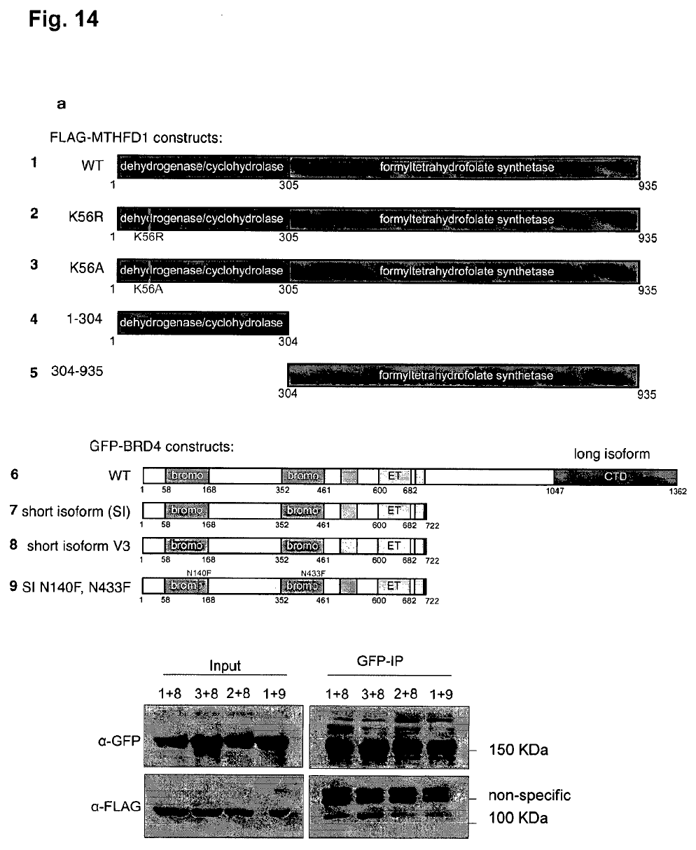

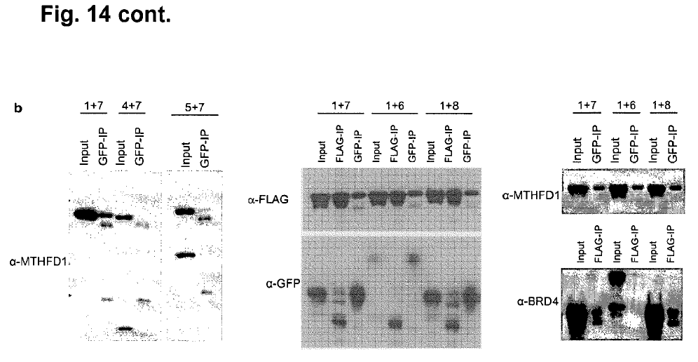

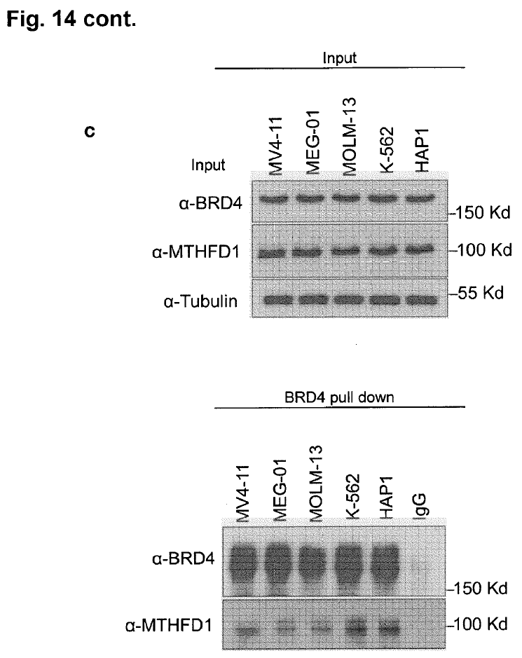

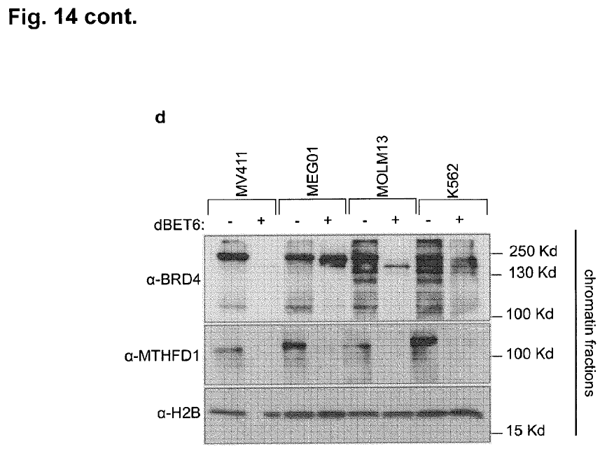

FIG. 14: (A) Upper panel: MTHFD1 and BRD4 constructs used in immunoprecipitation experiments. Lower panel: GFP immunoprecipitation from HEK293 cells overexpressing the indicated constructs shows increased interaction of MTHFD1(K56A) and decreased interaction of MTHFD1(K56R) with the short isoform of BRD4. Similarly, the interaction is impaired in the BRD4 double bromodomain mutant. (B) GFP immunoprecipitation from HEK293 cells overexpressing the indicated constructs shows interaction of BRD4 with full-length MTHFD1 but not with the individual domains of the protein. (C) Western blot confirmation of the BRD4-MTHFD1 interaction in leukemia cell lines. (D) Western blot from chromatin fractions of MEG-01, K-562, MV4-11 and MOLM-13 cells treated with dBET6 for 2 h.

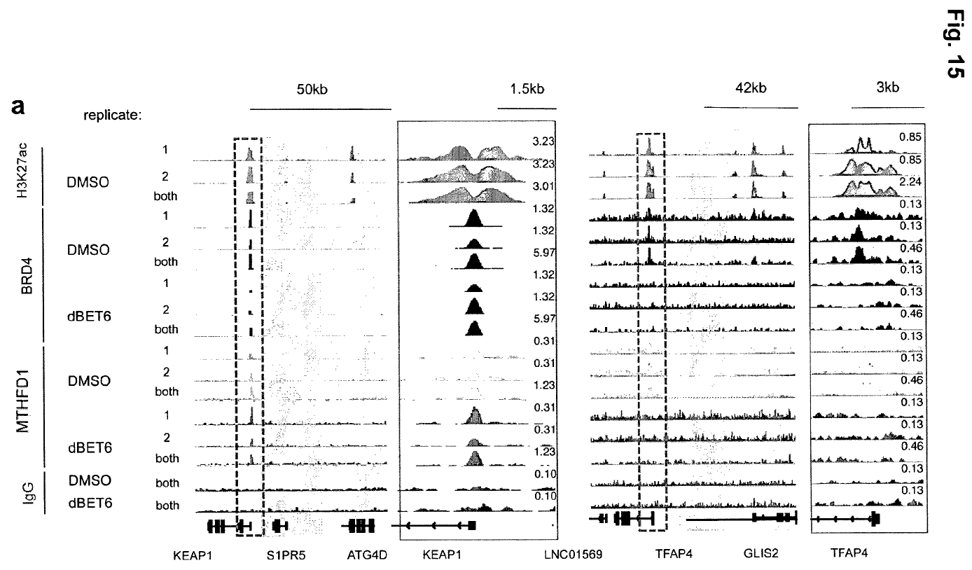

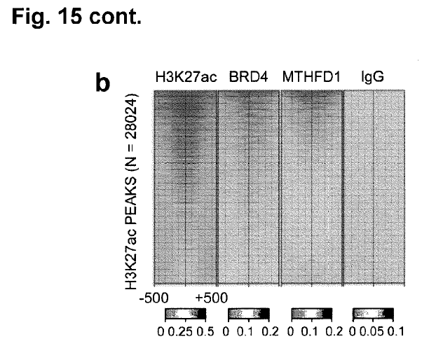

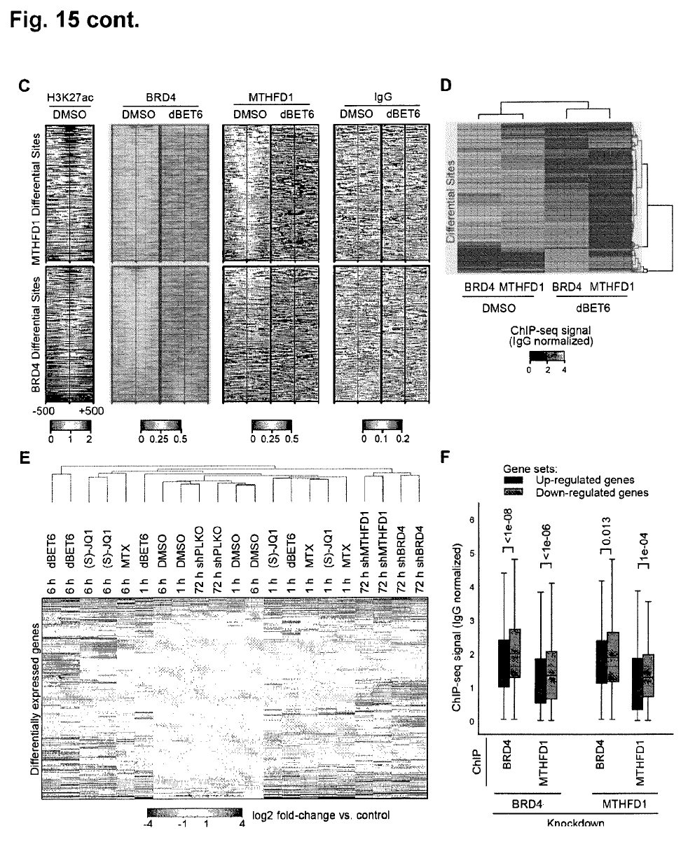

FIG. 15: (A) Representative genome browser view of BRD4 and MTHFD1 binding in the H3K27ac-marked promoters of KEAP1 (left) and TFAP4 (right). All ChIP tracks were normalized to total mapped reads and the respective IgG control was subtracted from the merged replicate tracks. (B) Enrichment of BRD4 and MTHFD1 ChIP signal in H3K27ac peaks. Peaks were sorted by H3K27ac abundance and data represent merged replicates in reads per basepair per million mapped reads. (C) Enrichment of BRD4 and MTHFD1 in the top 500 differentially bound sites between dBET6 and DMSO treatment. (D) Clustering of BRD4 and MTHFD1 abundance in the joint set of top 500 differentially bound sites between dBET6 or DMSO treatment. Hierarchical clustering with correlation as distance measurement was used. Values represent estimated factor abundance normalized by matched IgG signal. (E) Heatmaps of transcriptome analysis of HAP1 cells treated with 0.1 μM dBET6, 1 μM (S)-JQ1, 1 μM MTX, shRNAs targeting BRD4 or MTHFD1. Equal amount of DMSO, or non-targeting hairpins were used as respective control conditions. (F) Integration of ChIP-Seq and RNA-Seq data in HAP1 cells. BRD4 and MTHFD1 binding at sites associated with genes which are up- (dark grey) or down-regulated (light grey) upon knockdown of either BRD4 or MTHFD1. Values represent estimated factor abundance normalized by matched IgG signal and equality of distributions was assessed with the Kolmogorov-Smirnov test.

FIG. 16: (A) Illustrative genome browser views of BRD4 and MTHFD1 binding in the H3K27ac-marked promoters of KMT5A, BEND3, KMT2A, SKIDA1 (from left to right). All tracks were normalized by the total mapped reads in the genome and the respective IgG control was subtracted from the merged replicate tracks. Tracks for the same factor in different conditions were scaled similarly for comparison. Note the loss of BRD4 and MTHFD1 binding upon 1 μM dBET6 treatment for 2 hours. (B) Quantification of BRD4 and MTHFD1 in the top 500 differentially bound sites by MTHFD1 or BRD4 between dBET6 or DMSO treatment. Values represent estimated factor abundance normalized by matched IgG signal. Error bars represent 95th confidence interval. (C) Enriched motifs found in the joint set of regions with differential BRD4 or MTHFD1 binding upon dBET6 treatment. Note the recurrent “GGAA” motif found. (D) Reactome pathway terms enriched in genes bound differentially by BRD4 or MTHFD1 binding upon dBET6 treatment. The central heatmap illustrates which genes belong to each term. The enrichment score on the right represents “log(p-value)*Z-score”, where the Z-score is the gene set's deviation from an expected rank as defined by Enrichr. (E) Quality of ChIP-seq libraries through cross-correlation analysis. The X-axis represents an amount (in base pairs) by which the signal in two aligned strands is shifted by, and the Y-axis represents the cross-correlation between the signal in the strands at each shifted position. The first increase in cross-correlation (marked by a dark grey dashed line) is noise related with the read length used, and the second is true signal (light grey dashed line) related with enrichment of the immunoprecipitated protein and generally reflects the average length of DNA bound by the protein. The amount of baseline-normalized cross-correlation (NSC) and ratio between the two cross-correlation values (RSC) is indicative of signal-to-noise ratio and therefore of library quality (Qtag, increasing from 0 to 2).

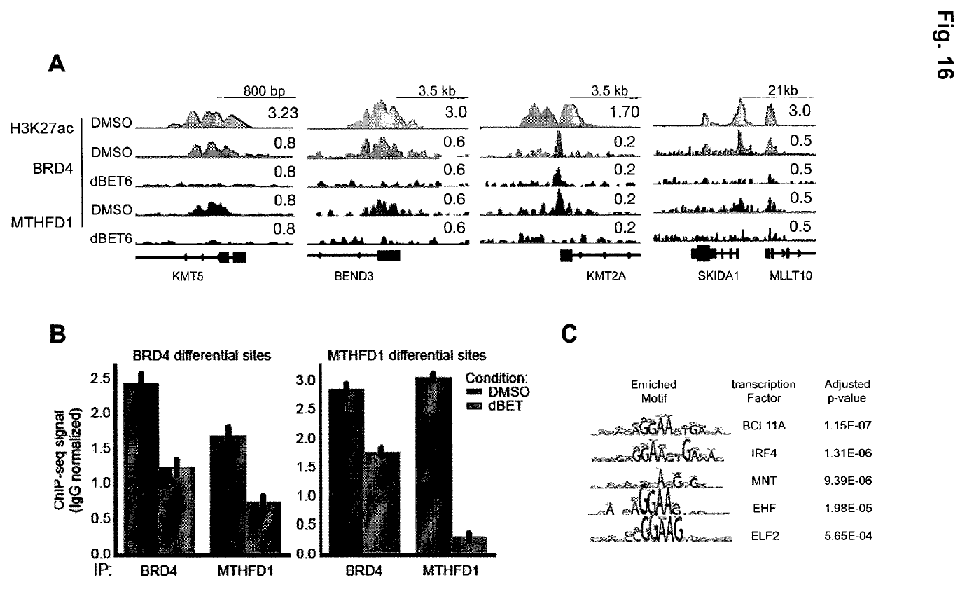

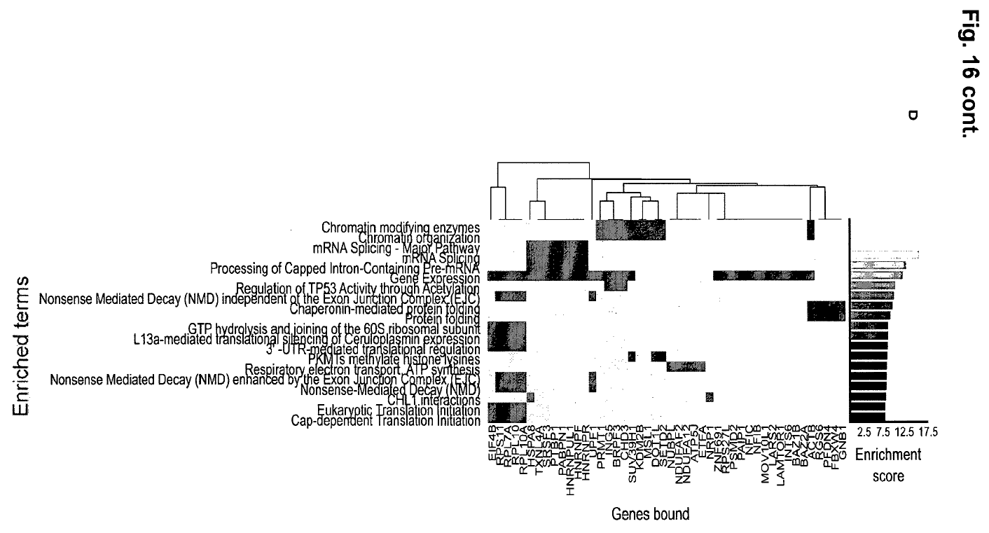

FIG. 17: (A) Heat map of relative transcriptional changes of HAP1 cells treated with 0.1 μM dBET6, 1 μM (S)-JQ1, 1 μM MTX, shRNAs targeting BRD4 or MTHFD1 alone or in combination. Equal amount of DMSO, or non-targeting hairpins were used as respective control conditions. (B) Integration of ChIP-Seq and RNA-Seq data in HAP1 cells. BRD4 and MTHFD1 binding at sites associated with genes which are up- (white) or down-regulated (black) upon knockdown of either BRD4, MTHFD1, or treatment with either JQ1 or Methotrexate. Binding in random sets of genes of the same size as the respective up- or down-regulated sets is displayed as control. Values represent estimated factor abundance normalized by matched IgG signal and equality of distributions was assessed with the Kolmogorov-Smirnov test.

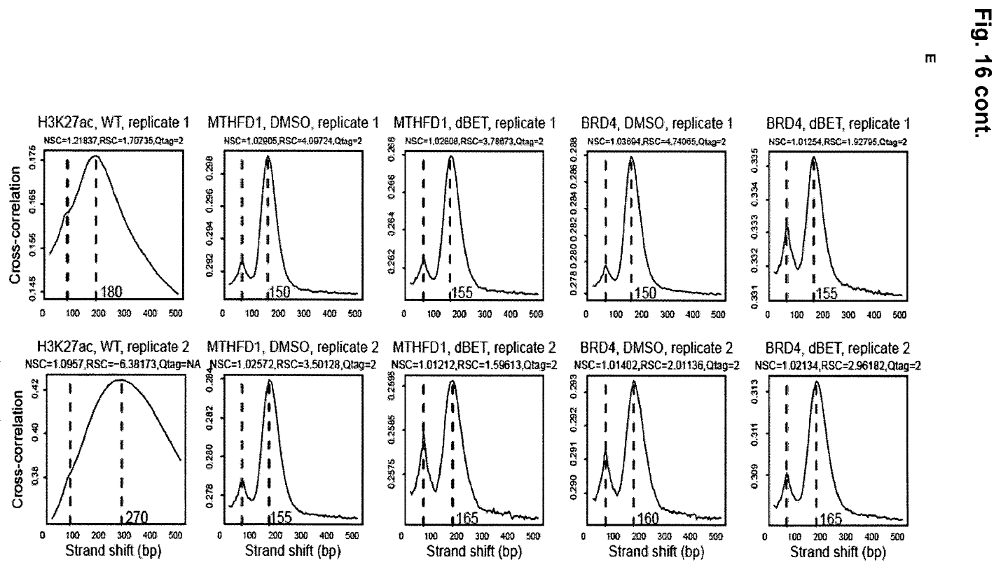

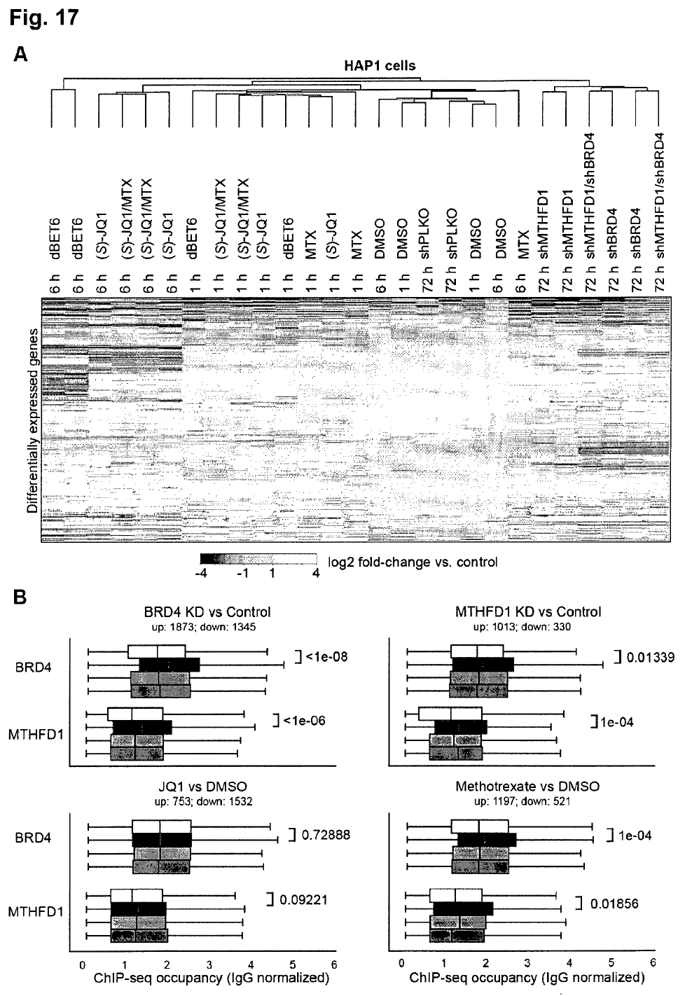

FIG. 18: (A) Heat map of relative transcription changes in K-562 cells treated with 0.1 μM dBET6, 1 μM (S)-JQ1, 1 μM MTX, shRNAs targeting BRD4 or MTHFD1 alone or in combination. Equal amount of DMSO, or non-targeting hairpins were used as respective controls. (B) Heat map matrix of relative transcription changes in A549 cells treated with 0.1 μM dBET6, 1 μM (S)-JQ1, 1 μM MTX, shRNAs targeting BRD4 or MTHFD1 alone or in combination. Equal amount of DMSO, or non-targeting hairpins were used as respective controls.

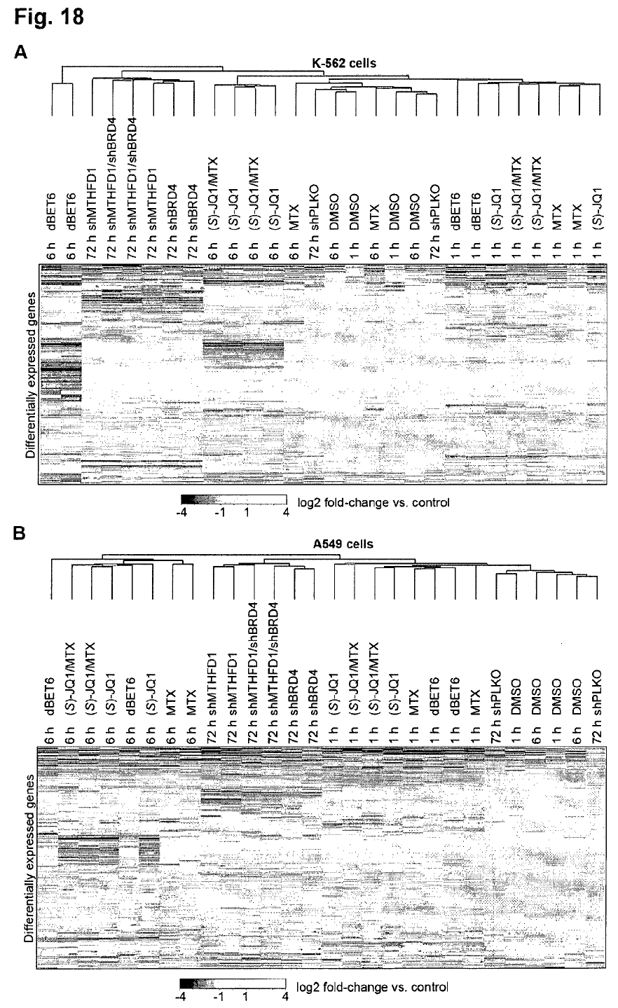

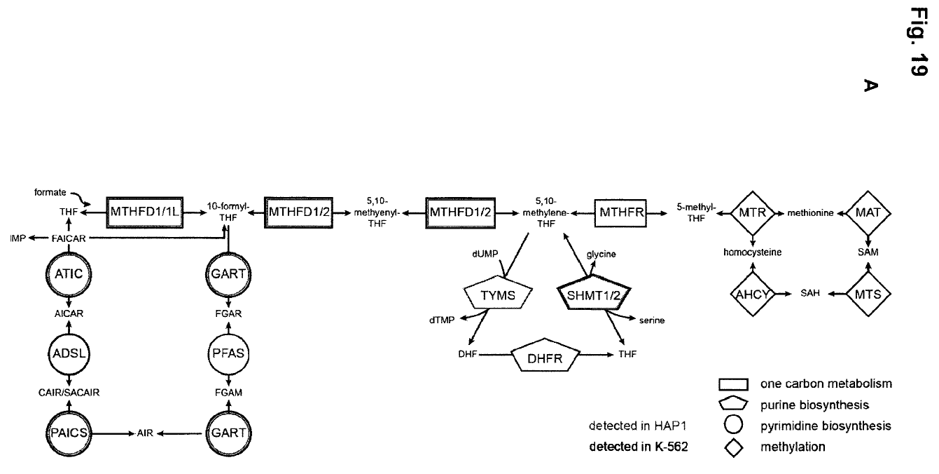

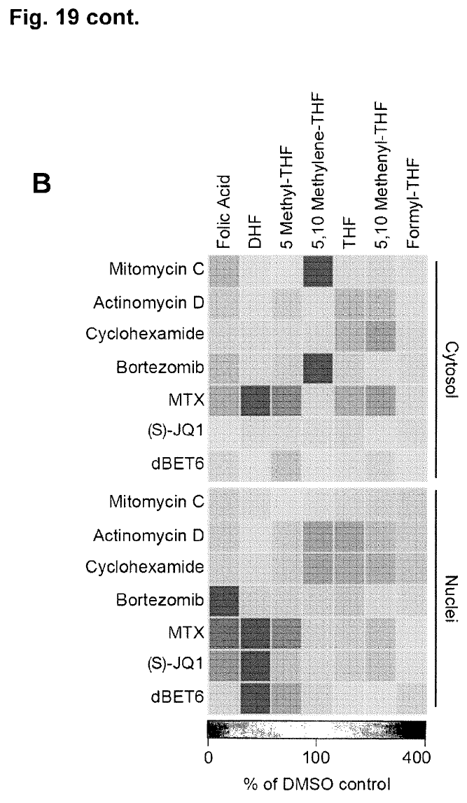

FIG. 19: (A) Representation of the folate pathway. Enzyme names are reported inside the geometric shapes, connecting the different metabolites. Enzymes that were found associated with chromatin in HAP1 and K-562 cells by mass spectrometry analysis are indicated in light grey and dark grey, respectively. Two biological replicates were done. (B) Heatmaps showing relative changes in folate metabolites levels in the of nuclear and cytosolic fraction of HAP1 cells treated with 1 μM of Mitomycin C, Actinomycin D, Bortexomib, MTX and (S)-JQ1, 0.5 μM of dBET6 or 12.5 μM of Cyclohexamide for 6 hours. Equal amount of DMSO was used as control, 2 biological replicates were done for each experimental condition.

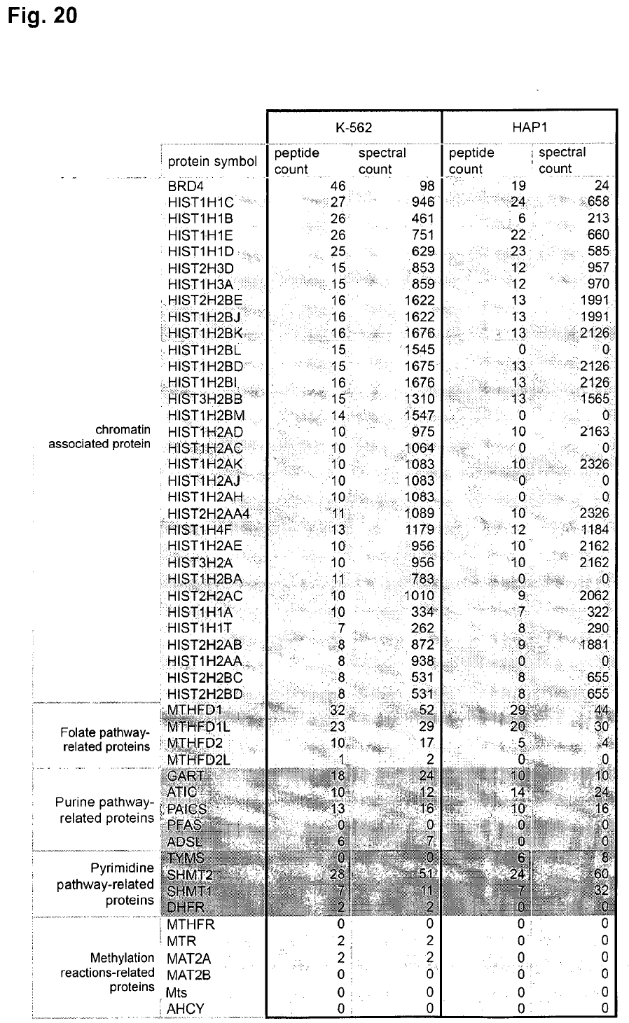

FIG. 20: Peptide and spectral counts identified by MS analysis of HAP1 chromatin extracts, Two biological replicates were done. Enzymes of the folate pathway found associated with chromatin are written in regular, while in italic are the “not-found”. BRD4 and histones are shown as control for chromatin associated proteins.

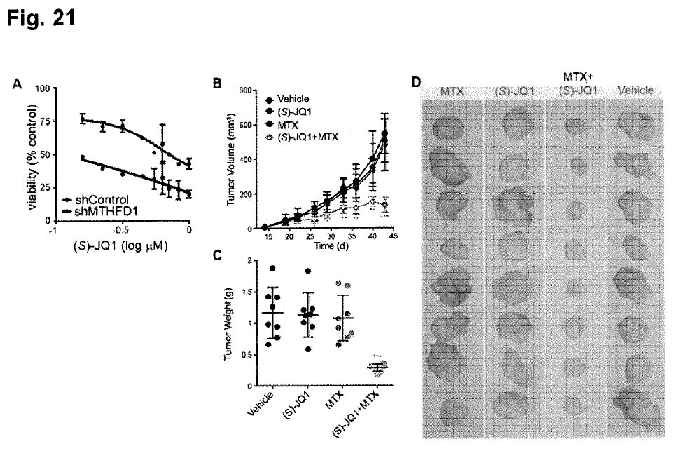

FIG. 21: (A) Knock-down of MTHFD1 in A549 cells followed by 72 hours treatment with increasing concentrations of (S)-JQ1. (B) Tumor volumes from a A549 xenograft mouse model treated five times per week with 30 mg/kg (S)-JQ1 and/or twice weekly with 25 mg/kg MTX from day 19. Means and standard deviations from eight mice per group. Asterisks indicate significance (* p<0.05; ** p<0.005; *** p<0.0001). (C) Weight and (D) images of tumors at the end of the experiment (day 43).

The invention will now be described by reference to the following examples which are merely illustrative and are not to be construed as a limitation of the scope of the present invention.

### EXAMPLES

Methods:

Cell Culture and Transfection

KBM7 (human chronic myelogenous leukemia cell lines), MV4-11 (biphenotypic B myelomonocytic leukemia), MEG-01 (human chronic myelogenous leukemia), K-562 (human chronic myelogenous leukemia) and HAP1 (KBM7-derived) cell lines were cultured in Iscove's Modified Dulbecco's Medium (IMDM, Gibco), supplemented with 10% Fetal Bovine Serum (FBS; Gibco). HEK293T (human embryonic kidney) and HELA (cervix adenocarcinoma) cell lines were cultured in Dulbecco's Modified Eagles Medium (DMEM, Gibco) supplemented with 10% FBS. MOLM-13 (human acute monocytic leukemia), NOMO-1 (human acute monocytic leukemia) and A549 (lung carcinoma) cell lines were cultured in RPMI-1640 (Roswell Park Memorial Institute, Gibco) supplemented with 10% FBS. All the mentioned cell lines were incubated in 5% CO₂ atmosphere at 37° C.

HEK293T cells were transfected with Lipofectamine 2000 (Invitrogen) according to the manufacturer's instructions.

The Retroviral gene trap vector (pGT-GFP; see below) was a gift from Dr. Sebastian Nijman, Group Leader at the Cell biology, Signaling, Therapeutics Program, Ludwig Cancer Research (Oxford, UK).

GFP-MTHFD1 plasmid was a gift from Professor Patrick Stover, Director of the Division of Nutritional Sciences, Cornell University (Ithaca, N.Y.).

Western Blot and Immunoprecipitation

For Western Blot, proteins were separated on polyacrylamide gels with SDS running buffer (50 mM Tris, 380 mM Glycine, 7 mM SDS) and transferred to nitrocellulose blotting membranes. All membranes were blocked with blocking buffer (5% (m/v) milk powder (BioRad) in TBST (Tris-Buffered Saline with Tween: 50 mM Tris (tris (hydroxymethyl)aminomethane), 150 mM NaCl, 0.05% (v/v) Tween 20, adjusted to pH 7.6). Proteins were probed with antibodies against BRD4 (ab128874, 1:1000, Abcam), Actin (ab16039, 1:1000, Abcam), MTHFD1 (ab70203, Abcam; H120, Santa Cruz; A8, Santa Cruz; all used at 1:1000), GFP (G10362, 1:1000, Life Technology), RCC1 (C-20, 1:1000, Santa Cruz), β-Tubulin (T-4026, 1:1000, Sigma), SHMT1 (ab186130, 1:1000, Abcam) and H2B (ab156197, 1:1000, Abcam) and detected by HRP (horseradish peroxidase) conjugated donkey anti-rabbit IgG antibody (ab16284, 1:5000, Abcam) or donkey anti-mouse IgG antibody (Pierce) and visualized with the Pierce ECL Western Blotting substrate (Amersham), according to the provided protocol.

For immunoprecipitation, 1 mg of protein extract was incubated overnight at 4° C. with 10 μl of Dynabeads (either A or G, Life technology) preincubated for 2 hours at 4° C. with 1 μg of BRD4 (ab128874, Abcam), MTHFD1 (A8, Santa Cruz) or GFP (G10362, Life Technology) antibodies.

Immunofluorescence and Live Cell Imaging

For immunofluorescence, cells were grown on coverslips precoated with Polylysine (Sigma). After the desired treatment, cells were washed with PBS and fixed with cold methanol for at least 24 hours. Blocking was performed in PBS/3% bovine serum albumin (BSA)/0.1% Triton for 30 minutes. Cells were then incubated with primary antibody for 30 minutes at room temperature (MTHFD1 H120, Santa Cruz; BRD4 ab128874, Abcam). After washing, they were incubated with secondary antibodies (Alexa Fluor 488 Goat Anti-Rabbit and Alexa Fluor 546 Donkey Anti-Mouse, Thermo Fisher Scientific) for 30 minutes in the dark. Finally, they were washed and incubated with DAPI (4,6-diamidino-2-phenylindole) for 5 minutes at room temperature in the dark. 3 PBS washing steps were done to remove the excess of antibodies and DAPI and coverslips were mounted with Propyl gallate (Sigma) on slides. Pictures were taken with a Leica DMI6000B inverted microscope and 63× oil objective and analyzed with Fiji (ImageJ).

Live cell imaging pictures were taken from cells seeded on clear flat bottom 96-well or 384-well plates (Corning), with the Operetta High Content Screening System (PerkinElmer), 20× objective and non-confocal mode. RFP quantification was done using the basic PerkinElmer software for nuclei detection and analysis, adapted for the nucleus diameter range of the specific cell line used (KBM7, 13 μm). Only RFP positive nuclei were detected and counted.

Cell Cycle Assay

For cell cycle analysis, 1 million cells were fixed with 70% ethanol for 24 hours, washed with PBS/1% BSA/0.1% Tween and incubated with RNase for 30 minutes. Nuclei were stained with 5 μg/ml PI (propidium iodide, Sigma) for 10 minutes prior to FACS analysis (BD FACSCalibur Flow Cytometer).

RNA Extraction and RT-PCR

RNA extraction was performed with TRIzol Reagent (Life Technologies) according to the standard protocol and Reverse Transcription (RT) was performed using the High Capacity cDNA Reverse Transcription Kit (Applied Biosystems).

QPCR was performed using the Power SYBR Green Master mix (Invitrogen) as described in the manufacture's protocol.

QPCR primers used:

Actin (Sigma; forward 5′-ATGATGATATCGCCGCGCTC,
reverse 5′-CCACCATCACGCCCTGG).
BRD1 (Sigma; forward 5′-GAAGAAGCAGTTTGTGGAGC,
reverse 5′-GCAGTCTCAGCGAAGCTCAC).
BRD2 (Sigma; forward 5′-GCTTGGGAAGACTTTGTTGG,
reverse 5′-TGTCAGTCACCAGGCAGAAG)
BRD3 (Sigma; forward 5′-AAGAGAAGGACAAGGAGAAGG,
reverse 5′-CTTCTTGGCAGGAGCCTTCT).
BRD4 (Sigma; forward 5′CAGGAGGGTTGTACTTATAGCA,
reverse 5′-CTACTGTGACATCATCAAGCAC).
BRDT (Sigma; forward 5′-TCAAAGATCCCGATTGAACC,
reverse 5′-CGGAAAGGTACTTGGGACAA)

Real-time amplification results were normalized to the endogenous housekeeping gene Actin. The relative quantities were calculated using the comparative CT (Cycle Threshold) Method (ΔΔCT Method).

Gene-Trap Genetic Screening

pGT-GFP contains an inactivated 3′ LTR, a strong adenoviral (Ad40) splice-acceptor site, GFP and the SV40 polyadenylation signal. Gene trap virus was produced by transfection of 293T cells in T150 dishes with pGT-GFP combined with retroviral packaging plasmids. The virus-containing supernatant was collected after 30, 48 and 72 hours of transfection and concentrated using ultracentrifugation for 1.5 hours at 24100 rpm in a Beckman Coulter Optima L-100 XP ultracentrifuge using an SW 32 Ti rotor.

REDS1 clone was mutagenized by infection of 24-well tissue culture dish containing 1 million cells per well using spin infection for 45 minutes at 2000 rpm. GT-infected cells were assessed by FACS to determine the percentage of infection (percentage of GFP positive cells). If such percentage was above 70%, REDS1 GFP/RFP double positive cells were sorted and left in culture for 2 weeks to get the proper amount of cells to use in the library preparation for sequencing.

Cell Sorting

RFP/GFP double positive cell sorting was performed using the FACSAria (BD Biosciences) sorter. Gates for positive or negative RFP or GFP populations were done using the appropriate positive or negative controls, RFP/GFP double positive cells were sorted 7 days after GT infection. RFP/GFP double positive cells were grown up to get the needed amount for DNA library preparation (30 millions).

DNA Library Preparation

DNA was extracted from 30 million GFP/RFP double positive REDS1 cells using the Genomic DNA isolation QIAamp DNA mini kit (Qiagen). 4 μg were digested with NlaIII or MseI (4 digestions each enzyme). After spin column purification (Qiagen), 1 μg of digested DNA was ligated using T4 DNA ligase (NEB) in a volume of 300 μl (total of 4 ligations). The reaction mix was purified and retroviral insertion sites were identified via an inverse PCR protocol adapted to next generation sequencing¹⁶.

FISH Assay

The RFP specific probe (RFP_probe) was PCR performed using RFP specific primers (Sigma; forward 5′-CGGTTAAAGGTGCCGTCTCG, reverse 5′-AGGCTTCCCAGGTCACGATG) and labeled using dig-dUTP (DIG Nick Translation Mix, Roche). The FISH assay procedure was performed as previously described¹⁵.

AlphaLISA Assay

The Amplified Luminescent Proximity Homogenous Assay (AlphaLISA©), a homogenous and chemiluminescence-based method, was performed to explore the direct interaction of BRD4 and acetylated substrates.

Briefly, in this assay, the biotinylated MTHFD1 acetylated peptides (possible substrates) are captured by streptavidin-coupled donor beads. GST-tagged BRD4 (produced as previously described¹⁵) is recognized and bound by an anti-GST antibody conjugated with an acceptor bead. In case of interaction between BRD4 and one acetylated peptide, the proximity between the partners (<200 nm) allows that the excitation (680 nm wavelength) of a donor bead induces the release of a singlet oxygen molecule (¹O₂) that then triggers a cascade of energy transfer in the acceptor bead, resulting in a sharp peak of light emission at 615 nm.

GST-BRD4 and each of the MTHFD1 acetylated peptides were incubated together. After 30 minutes, GSH (Glutathione) Acceptor beads (PerkinElmer) were added and after another incubation time of 30 minutes, Streptavidin-conjugated donor beads (PerkinElmer) were added. The signal (alpha counts) was read by the EnVision 2104 Multilabel Reader (PerkinElmer).

Preparation of Nuclear Cell Extracts for Proteomics

Nuclear extract was produced from fresh cells grown at 5.0×10e6 cells/mL. Cells were collected by centrifugation, washed with PBS and resuspended in hypotonic buffer A (10 mM Tris-Cl, pH 7.4, 1.5 mM MgCl₂, 10 mM KCl, 25 mM NaF, 1 mM Na₃VO₄, 1 mM DTT, and 1 Roche protease inhibitor tablet per 25 ml). After ca. 3 min cells were spun down and resuspended in buffer A and homogenized using a Dounce homogenizer. Nuclei were collected by centrifugation in a microfuge for 10 min at 3300 rpm, washed with buffer A and homogenized in one volume of extraction buffer B (50 mM Tris-Cl, pH 7.4, 1.5 mM MgCl₂, 20% glycerol, 420 mM NaCl, 25 mM NaF, 1 mM Na₃VO₄, 1 mM DTT, 400 Units/ml DNase I, and 1 Roche protease inhibitor tablet per 25 ml). Extraction was allowed to proceed under agitation for 30 min at 4° C. before the extract was clarified by centrifugation at 13000 g. The extract was diluted 3:1 in buffer D (50 mM Tris-Cl, pH 7.4 (RT), 1.5 mM MgCl₂, 25 mM NaF, 1 mM Na₃VO₄, 0.6% NP40, 1 mM DTT, and Roche protease inhibitors), centrifuged again, and aliquots were snap frozen in liquid nitrogen and stored at −80° C.

Immunopurification (IP-MS)

Anti-BRD4 (A301-985A, Bethyl Labs) antibody (50 μg) was coupled to 100 μl AminoLink resin (Thermo Fisher Scientific). Cell lysate samples (5 mg) were incubated with prewashed immuno resin on a shaker for 2 h at 4° C. Beads were washed in lysis buffer containing 0.4% Igepal-CA630 and lysis buffer without detergent followed by two washing steps with 150 mM NaCl.

Samples were processed by on-bead digest with Lys-C and Glycine protease before they were reduced, alkylated and digested with Trypsin.

NanoLC-MS Analysis

The nano HPLC system used was an UltiMate 3000 HPLC RSLC nano system (Thermo Fisher Scientific, Amsterdam, Netherlands) coupled to a Q Exactive mass spectrometer (Thermo Fisher Scientific, Bremen, Germany), equipped with a Proxeon nanospray source (Thermo Fisher Scientific, Odense, Denmark).

The Q Exactive mass spectrometer was operated in data-dependent mode, using a full scan (m/z range 350-1650, nominal resolution of 70 000, target value 1E6) followed by MS/MS scans of the 12 most abundant ions. MS/MS spectra were acquired using normalized collision energy 30%, isolation width of 2 and the target value was set to 5E4. Precursor ions selected for fragmentation (charge state 2 and higher) were put on a dynamic exclusion list for 30 s. Additionally, the underfill ratio was set to 20% resulting in an intensity threshold of 2E4. The peptide match feature and the exclude isotopes feature were enabled.

Data Analysis

For peptide identification, the RAW-files were loaded into Proteome Discoverer (version 1.4.0.288, Thermo Scientific). All hereby created MS/MS spectra were searched using Mascot 2.2.07 (Matrix Science, London, UK) against the human swissprot protein sequence database. The following search parameters were used: Beta-methylthiolation on cysteine was set as a fixed modification, oxidation on methionine. Monoisotopic masses were searched within unrestricted protein masses for tryptic peptides. The peptide mass tolerance was set to ±5 ppm and the fragment mass tolerance to ±30 mmu. The maximal number of missed cleavages was set to 2. For calculation of protein areas Event Detector node and Precursor Ions Area Detector node, both integrated in Thermo Proteome Discoverer, were used. The result was filtered to 1% FDR using Percolator algorithm integrated in Thermo Proteome Discoverer. Additional data processing of the triplicate runs including label-free quantification was performed in MaxQuant using the Andromeda search engine applying the same search parameters as for Mascot database search. For subsequent statistical analysis Perseus software platform was used to create volcano plots, heat maps and hierarchical clustering.

ChIPmentation

ChIPmentation experiments were performed as described in Schmidl et al., Nature Methods 2015¹⁷. ChIP-Seq Sample Preparation

Three 15 cm dishes with cells at 70-80% of confluency were used for one ChIP experiment. Briefly, cells were cross-linked with 1% formaldehyde for 10 minutes at room temperature, and then quenched with 125 mM glycine for 5 minutes at room temperature. Then, cells were washed with cold PBS, collected in 15 ml tubes and washed again with cold PBS by centrifugation at 1200 rpm for 5 minutes at 4° C. and finally snap frozen.

ChIP was performed as described¹⁸ by using BRD4 (Bethyl Laboratories, Inc.) and MTHFD1 (sc-271413, Santa Cruz) antibodies. In brief, crosslinked cell lysates were sonicated in order to shred the chromatin into 200-500 bp fragments. Fragmented chromatin was incubated overnight at 4° C. with antibodies, followed by 2 hours at 4° C. with pre-blocked Dynabeads Protein G (ThermoFisher Scientific). Beads were washed twice with low salt buffer, twice with high salt buffer, twice with LiCl buffer, twice with 1× TE buffer and finally eluted with elution buffer for 20 min at 65° C. The elution products were treated with RNaseA for 30 minutes at 37° C., followed by proteinase K treatment at 55° C. for 1 hour, and then incubated at 65° C. overnight to reverse the crosslinks. The samples were further purified by using a PCR purification kit (Qiagen). ChIP-seq libraries were sequenced by the Biomedical Sequencing Facility at CeMM using the Illumina HiSeq3000/4000 platform and the 50-bp single-end configuration.

ChIP-Seq Data Analysis

Reads containing adapters were trimmed using Skewer¹⁹ and aligned to the hg19/GRCh37 assembly of the Human genome using Bowtie2²⁰ with the “—very-sensitive” parameter and duplicate reads were marked and removed with sambamba. Library quality was assessed with the phantomPeakQualtools scripts²¹. For visualization exclusively, the inventors generated genome browser tracks with the genomeCoverageBed command in BEDTools²² and normalized such that each value represents the read count per base pair per million mapped and filtered reads. This was done for each sample individually and for replicates merged. In visualizations, the inventors simply subtracted the respective merged control IgG tracks from each merged IP using IGV²³. They used HOMER findPeaks²⁴ in “factor” mode to call peaks on both replicates with matched IgG controls as background and used DiffBind²⁵ to detect differential binding of BRD4 or MTHFD1 in H3K27ac peaks dependent on dBET6 treatment. The top 500 differential regions for each comparison (sorted by p-value) were used for visualization using SeqPlots²⁶ and clustering with using the concentration values of each factor in each condition estimated with DiffBind. The same top differential regions were input into Enrichr²⁷ as BED files and enrichments for Reactome pathway were retrieved.

Molecular Modeling

For calculating the binding affinity of MTHFD1(K56ac) towards BRD4, six crystal structures of BRD4 co-crystallized with any peptide were downloaded from the RCSB Protein Databank (PDB; www.rcsb.org)²⁸. The X-ray structures were prepared using the QuickPrep protocol of the MOE software package. With that, hydrogens and missing atoms were added, charges were calculated, protonation states optimized and clashes and strain were removed by performing a short energy minimization. Prior to mutating the co-crystallized peptide into the MTHFD1(K56ac), the crucial interaction of the acetylated Lys with Asn140 was restrained. The virtual mutations as well as the affinity and stability calculations were performed using the Protein Design tools (Residue Scan with default settings) of the MOE software package.

For predicting the binding of Methotrexate (MTX) to MTHFD1 (acetylated and unacetylated), the X-ray structure 1A4I was prepared with the QuickPrep protocol of MOE. As the binding pocket of 1A4I is highly solvated, water molecules might interfere with MTX binding during the docking run. Therefore, water molecules were removed for all calculations. For the comparison of binding acetylated vs unacetylated MTHFD1, the prepared crystal structure was acetylated using the Protein Builder in MOE, followed by a short energy minimization of the mutated residue. Furthermore, MTX was prepared and protonated in MOE. A conformational analysis using the LowModeMD method with default settings provided 37 different MTX conformations. These 37 conformations were docked into the acetylated and unacetylated structures of MTHFD1 using the induced fit docking protocol in MOE with default settings.

Interaction fingerprints of the docked structures were calculated using the PLIF tool in MOE.

Chromatin Purification and LC-MS/MS Analysis

Cell fractionation and chromatin enrichment was carried out as previously described²⁹ with some adaptations. Briefly, for 100 million cells, the chromatin enriched pellet was taken up in 250 μl benzonase digestion buffer (15 mM HEPES, 1 mM EDTA, 1 mM EGTA, 0.1% NP40, protease inhibitor cocktail (cOmplete, Roche)) after washing, and sonicated for 120 seconds on the Covaris S220 focused-ultrasonicator with the following settings: Peak Power 140; Duty-Factor 10.0; Cycles/Burst 200. After addition of 0.25 U benzonase and 2.5 μg RNase, the chromatin was incubated for 40 minutes at 4° C. on a rotary shaker. 2× SDS lysis buffer (100 mM HEPES, 4% SDS, 2 mM PMSF and protease inhibitor cocktail (cOmplete, Roche)) was added to the samples in a 1:1 ratio and incubated for 10 minutes at room temperature followed by 5 minutes denaturation at 99° C. After centrifugation at 16,000 g for 10 minutes at room temperature, the supernatant was transferred to a new tube. MS sample preparation was performed using the FASP protocol as previously described³⁰. Reverse-phase chromatography at high and low pH was performed for two-dimensional peptide separation prior to MSMS analysis. Peptides were purified using solid-phase extraction (SPE) (MacroSpin Columns, 30-300 μg capacity, Nest Group Inc. Southboro, Mass., USA) and reconstituted in 23 μL 5% acetonitrile, 10 mM ammonium formate. An Agilent 1200 HPLC system (Agilent Biotechnologies, Palo Alto, Calif.) equipped with a Gemini-NX C18 (150×2 mm, 3 μm, 110 Å, Phenomenex, Torrance, US) column was used for the first dimension of liquid chromatography. Peptides were separated into 20 time based fractions during a 30 min gradient ranging from 5 to 90% acetonitrile containing 10 mM ammonium formate, pH 10, at a flow rate of 100 μL/min. Samples were acidified by the addition of 5 μL 5% formic acid. Solvent was removed in a vacuum concentrator, and samples were reconstituted in 5% formic acid. Mass spectrometric analyses were performed on a Q Exactive mass spectrometer (ThermoFisher, Bremen, Germany) coupled online to an Agilent 1200 series dual pump HPLC system (Agilent Biotechnologies, Palo Alto, Calif.). Samples were transferred from the thermostatted autosampler (4° C.) to a trap column (Zorbax 300SB-C18 5 μm, 5×0.3 mm, Agilent Biotechnologies, Palo Alto, Calif., USA) at a constant flow rate of 45 μL/min. Analyte separation occurred on a 20 cm 75 μm inner diameter analytical column, that was packed with Reprosil C18 (Dr. Maisch, Ammerbuch-Entringen, Germany) in house. The 60-minute gradient ranged from 3% to 40% organic phase at a constant flow rate of 250 nL/min. The mobile phases used for the HPLC were 0.4% formic acid and 90% acetonitrile plus 0.4% formic acid for aqueous and organic phase, respectively. The Q Exactive mass spectrometer was operated in data-dependent mode with up to 10 MSMS scans following each full scan. Previously fragmented ions were dynamically excluded from repeated fragmentation for 20 seconds. 100 ms and 120 ms were allowed as the maximum ion injection time for MS and MSMS scans, respectively. The analyzer resolution was set to 70,000 for MS scans and 35,000 for MSMS scans. The automatic gain control was set to 3×106 and 2×105 for MS and MSMS, respectively, to prevent the overfilling of the C-trap. The underfill ratio for MSMS was set to 6%, which corresponds to an intensity threshold of 1×105 to accept a peptide for fragmentation. Higher collision energy induced dissociation (HCD) at a normalized collision energy (NCE) of 34 was employed for peptide fragmentation and reporter ion generation. The ubiquitous contaminating siloxane ion Si(CH3)2O)6 was used as a single lock mass at m/z 445.120024 for internal mass calibration.

MS Data Analysis (Chromatin Fraction)

The acquired raw MS data files were processed as previously described³¹. The resultant peak lists were searched against the human SwissProt database version 20150601 with the search engines Mascot (v.2.3.02) and Phenyx (v.2.5.14).

For TMT quantitation the isobar R package was used³². As the first step of the quantitation, the reporter ion intensities were normalized in silico to result in equal median intensity in each TMT reporter channel. Isobar statistical model considers two P-values: P-value sample that compares the abundance changes due to the treatment to the abundance changes seen between biological replicates and P-value ratio that models for noise/variability in mass spectrometry data collection. P-value ratio was further corrected for false discovery rate (FDR). Abundance of a protein was considered to be changed significantly if both P-value sample and FDR corrected P-value ratio were less than 0.05.

Preparation of Nuclear Cell Extracts for Metabolomics

Nuclei were extracted by hypotonic lysis. Briefly, intact cells treated (as indicated in the results section) were washed twice with cold PBS and incubated on ice for 10 minutes with hypotonic lysis buffer (10 mM HEPES, pH 7.9, with 1.5 mM MgCl₂, 10 mM KCl and protease inhibitor cocktail (cOmplete, Roche); buffer-cells volume ratio 5:1). Pellet was gently resuspended three times during the incubation. Nuclei were collected by centrifugation (420 g×5 minutes) and immediately snap frozen.

The metabolomic assay and data analysis was performed by Metabolomic Discoveries (http://www.metabolomicdiscoveries.com; Germany). Briefly, LC-QTOF/MS-based non-targeted metabolite profiting was used to analyze nuclear metabolites in the range of 50-1700 Da, with an accuracy up to 1-2 ppm and a resolution of mass/Δmass=40,000. Metabolites measured in the LC are annotated according to their accurate mass and subsequent sum formula prediction. Metabolites that were not annotated in the LC-MS-analyses are listed according to their accurate mass and retention time.

Metabolite Set Enrichment Analysis

Metabolite set enrichment analysis (MSEA)³³ was performed using the online tool MetaboAnalyst³⁴ (http://www.metaboanalyst.ca/). Briefly, for each pre-defined functional group a fold-change is computed between the observed number of significantly altered metabolites (considering both up- and down-regulation, t-test with p-value <0.05) and random expectation, as well as a corresponding pvalue (using Fisher's exact test).

Folate Extraction and LC MS/MS Analysis

In order to quantify folates in the nuclear and cytosolic fractions, 20 millions of HAP1 cells per condition were washed twice with cold PBS, and collected into 50 ml falcon tube by centrifugation for 5 minutes at 280 g and 4° C. Cell lysis was performed on ice in the dark by incubating cell pellets with 1:5 hypotonic lysis buffer for 10 minutes. Nuclei were collected by centrifugation for 5 minutes at 420 g and 4° C. Supernatants (cytosolic fractions) were also collected. Both fractions were immediately snap frozen.

For nucleus samples, 10 μL of ISTD mixture was added to nucleus pellet in 1.5 mL Eppendorf tube followed by addition of 145 μL of ice-cold extraction solvent (10 mg/mL ascorbic acid solution in 80% methanol, 20% water, v/v). The samples were vortexed for 10 seconds, afterwards incubated on ice for 3 min and vortexed again for 10 seconds. After centrifugation (14000 rpm, 10 min, 4° C.), the supernatant was collected into HPLC vials. The extraction step was repeated and combined supernatants were used for LC-MS/MS analysis.

For cytoplasm samples, 10 μL of ISTD mixture was added to 75 μL of cytoplasm 1.5 mL Eppendorf tube followed by addition of 215 μL of ice-cold extraction solvent (10 mg/mL ascorbic acid solution in 80% methanol, 20% water, v/v). The samples were vortexed for 10 seconds, afterwards incubated on ice for 3 min and vortexed again for 10 seconds. After centrifugation (14000 rpm, 10 min, 4° C.), the supernatant was collected into HPLC vials and used for LC-MS/MS analysis.

An Acquity UHPLC system (Waters) coupled with Xevo TQ-MS (Waters) triple quadrupole mass spectrometer was used for quantitative analysis of metabolites. The separation was conducted on an ACQUITY HSS 13, 1.8 μm, 2.1×100 mm column (Waters) equipped with an Acquity HSS T3 1.8 μM Vanguard guard column (Waters) at 40° C. The separation was carried out using 0.1% formic acid (v/v) in water as a mobile phase A, and 0.1% formic acid (v/v) in methanol as a mobile phase B. The gradient elution with a flow rate 0.5 mL/min was performed with a total analysis time of 10 min. The autosampler temperature was set to 4° C. For detection, Waters Xevo TQ-MS in positive electrospray ionization mode with multiple reaction mode was employed. Quantification of all metabolites was performed using MassLynx V4.1 software from Waters. The seven-point linear calibration curves with internal standardization and 1/× weighing was constructed for the quantification.

Mouse Xenograft Studies

Mouse xenograft studies were performed as described previously³⁵. 2×106 A549 cells, diluted 1:1 in matrigel, were transplanted subcutaneously into NOD SCID gamma mice. Treatment (30 mg/kg (S)-JQ1 by intraperitoneal injection five times per week, and 25 mg/kg MTX per intraperitoneal injection twice weekly) was started when tumors were established, 19 days post transplantation. Tumor volumes were evaluated twice a week by measuring two perpendicular diameters with calipers. Tumor volume was calculated using the following equation: (width*width*length)/2. Treatment was performed according to an animal licence protocol approved by the Bundesministerium für Wissenschaft and Forschung (BMWF-66.009/0280-II/3b/2012). At day 43 mice were sacrificed and tumors excised and weighted.

A Genetic Loss-of-Function Screen for BRD4 Pathway Genes

The prerequisite for effective GT genetic screens is a haploid system where monoallelic disruptive GT integration results in gene knock-out (KO). Therefore, KBM7 cells, a chronic myeloid leukemia cell line with near haploid karyotype, were chosen for the generation of the BRD4 reporter cell lines as previously described¹⁵. The inhibition of BRD4 with the potent inhibitor (S)-JQ1 led to rapid and consistent expression of the reporter gene red fluorescent protein (RFP) in REDS, which could easily be detected by FACS (see FIG. 6A). Propidium iodide (PI) incorporation and FACS analysis were used to assay the cell cycle profile of several REDS clones in order to select a haploid clone suitable for GT-based genetic screen. Surprisingly, most of the originally selected clones showed increased, likely diploid, DNA content. REDS1 was the only clonal cell line with haploid karyotype (see FIG. 6B), also confirmed by metaphase spreads (see FIG. 6C). This finding indicated that the treatment with BRD4 inhibitors can induce a diploid-like phenotype in this specific cell line. To test the kinetics of this diploidization, WT (wild type)-KBM7 cells were treated overnight, either with DMSO, (S)-JQ1 or its inactive enantiomer (R)-JQ1 for one week and assessed the cell cycle profile by PI incorporation and FACS. Only (S)-JQ1 treatment induced WT-KBM7 diploidization (see FIG. 6D), therefore confirming the hypothesis of BRD4 inhibition-mediated effects on chromosome number, possible through chromosomal instabilities, chromosome segregation defects, or increased endoreplication.

The suitability of the REDS1 clone for a GT-based genetic screen was then further validated. The clone harbors a single genomic RFP integration as determined by fluorescence in situ hybridization (see FIG. 7A). REDS1 cells treated with 1 μM (S)-JQ1 for 24 hours robustly expressed RFP, which could be detected by live cell imaging (see FIG. 7B). As (S)-JQ1 potently inhibits other BET (bromodomain and extraterminal domain) proteins, short hairpin RNA (shRNAs) against BRD1, BRD2, BRD3, BRDT and BRD4 were tested for their ability to induce RFP expression. All hairpins caused >70% downregulation of their respective mRNA (see FIG. 7C). The RFP expression quantified from live cell imaging showed that only the downregulation of BRD4 induced an obvious increase of this parameter (see FIG. 7D). Finally, using a sequencing approach the RFP integration site was located in the first intron of the CDKAL1 gene on chromosome 6 (see FIG. 7E).

With the REDS1 clone validated, a GT-mediated genetic screen was performed in order to identify new functional partners for BRD4 (see FIG. 1A). The high specificity of the screening system relies on a direct read out (RFP signal) which clearly indicates chromatin remodeling in a BRD4 inhibition-like pattern. Therefore, the expression of RFP upon a specific gene KO indicates that such gene is involved in the chromatin remodeling at BRD4-dependent loci. Following infection of REDS1 cells with a GT virus carrying a green fluorescent protein (GFP) reporter gene, double positive cells (RFP⁺/GFP⁺) were sorted, as this cells population phenocopies BRD4 inhibition upon the KO of a specific gene (see FIG. 1B). Following the extraction of genomic DNA from this population, GT integration sites were amplified, sequenced and mapped onto the genome. Data were analysed for the number of independent integrations compared to an unselected control population and the distribution of disruptive sense vs. antisense integration of the GT vector. Three prominent hits emerged from this analysis, the long non coding RNA AC113189.5, methylenetetrahydrofolate dehydrogenase 1 (MTHFD1) and mediator of DNA damage checkpoint 1 (MDC1) (see FIGS. 1C, 8A and 11). Of these three genes, only MDC1, a gene involved in DNA repair, has previously been linked indirectly to BRD4 biology through the role of the short isoform of BRD4 as DNA insulator during DNA damage signaling. To validate MTHFD1 as genetic interactor of BRD4, REDS1 cells were treated with three different shRNAs resulting in 44-92% knock-down of MTHFD1 (see FIG. 1D). All three hairpins induced reporter RFP expression, and the effect size correlated with their knock-down efficiency (see FIGS. 1E and 1F). To rule out clone effects, the same experiment was repeated in the diploid REDS3 cells and similar results were obtained (see FIGS. 8B, 8C and 8D).

MTHFD1 is Recruited to Chromatin by Physical Interaction with BRD4

To understand the role of MTHFD1 in BRD4-mediated gene regulation, it was tested whether these two proteins interacted physically. Therefore, HEK293T cells were transfected with a plasmid encoding for GFP-MTHFD1. After 48 hours, GFP pull-down (PD) was performed and showed that BRD4 could co-immunoprecipitate (co-IP) with overexpressed (OE) MTHFD1 (see FIG. 9A). Similarly, BRD4 PD in HeLa cells showed that MTHFD1 interacted with the endogenous form of this bromodomain containing protein (see FIG. 9B). As BRD4 has been broadly studied for its role driving leukemia progression, an unbiased proteomic approach was used to identify all BRD4 interactors in K562, MOLM-13, MV4-11 and MEG01 cell lines (see FIGS. 2A and 9C). Only 13 proteins commonly interacted with BRD4 in all four cell lines. This set comprised several chromatin proteins like BRD3, LMNB1 and SMC3. In addition, MTHFD1, the folate pathway enzyme identified in the genetic screen as key factor required for BRD4 function, was identified in all four cell lines as direct interactor of BRD4. The interaction between MTHFD1 and BRD4 was also confirmed in pull-down experiments performed in K562, MOLM-13, MV4-11 and MEG01 cell lines used in the proteomic approach (see FIG. 14A).

While BRD4 is localized almost exclusively to the nucleus, the folate biosynthesis is considered to occur in the cytoplasm and mitochondria. However, recently SUMOylation dependent nuclear import of folate pathway enzymes has been described³⁶⁻³⁹. Nuclear vs cytosolic fractionation of HAP1, KBM7 and HEK293T cells indicated that MTHFD1 can be detected in the nucleus in all three cell lines (see FIG. 2B, upper panel). In contrast to another folate pathway enzyme, serine hydroxymethyltransferase 1 (SHMT1), nuclear MTHFD1 did not show any molecular weight changes, indicating that other mechanisms than SUMOylation were driving its nuclear localization. To further confirm this finding, HAP1 cells were treated with ginkgolic acid (GA), a small molecule inhibitor of SUMOylation, which did not cause a redistribution of MTHFD1 between the nucleus and cytoplasm (see FIG. 9D). It was next tested in which cellular compartment the interaction between BRD4 and MTHFD1 occurred. Therefore, MTHFD1 PD was performed in the cytosolic and nuclear fractions of HAP1, KBM7 and HEK293T cells. These experiments revealed that the interaction with BRD4 was happening exclusively in the nucleus (see FIG. 2B, lower panel). Given that BRD4 binds to acetylated proteins, particularly histones, with its bromodomains, the inventors aimed at understanding whether this mechanism of binding is also driving the interaction with MTHFD1. Interestingly, seven lysines on the MTHFD1 surface are known to be acetylated from proteomics studies, Synthetic acetylated MTHFD1 peptides (see FIG. 9E) were therefore used to perform an alphaLISA assay for their binding to GST-BRD4. Remarkably, one of the acetylated peptides, MTHFD1(47-66)K56ac, showed almost 5-fold increase of the alphaLISA signal when used at high concentration (see FIG. 9F). Moreover, the interaction occurred in a dose-responsive manner (see FIG. 9G), indicating that the acetylation of MTHFD1-K56 enhanced the binding to BRD4. A cheminformatics approach predicted that the MTHFD1 K56ac peptide bound the BRD4 bromodomains comparably or better than acetylated histone peptides (see FIG. 9H). However, the incubation of HAP1 cell lysate with the acetylated MTHFD1 peptide during the IP procedure was not able to inhibit the BRD4-MTHFD1 interaction, indicating that the stabilization of the interaction may depend on additional domains or other factors. Similarly, the BRD4-MTHFD1 interaction was unaffected by pharmacological inhibitors for BRD4 ((S)-JQ1) or MTHFD1 (methotrexate (MTX)) (see FIG. 9I). With the nucleus confirmed as the interaction site of BRD4 and MTHFD1, the inventors wanted to elucidate whether the BRD4-MTHFD1 complex was chromatin-bound or rather found in the soluble nuclear faction. In order to test if the acetylation of K56 of MTHFD1 was responsible for the interaction between MTHFD1 and BRD4 in cells, the inventors co-transfected HEK293-T cells with either FLAG-MTHFD1 WT, FLAG-MTHFD1(K56A) (which mimics the uncharged acetylated state), or FLAG-MTHFD1(K56R) (mutation of the same residue to a changed arginine) together with GFP-BRD4 WT. The MTHFD1(K56A) mutation enhanced interaction with BRD4, while MTHFD1(K56R) reduced the interaction. Consistently, they also proved that the double bromodomain mutant GFP-BRD4 N140F/N433F showed drastically reduced binding to FLAG-MTHFD1, when these two constructs were overexpressed together in HEK293-T cells see (FIG. 14B). Moreover, in cellular pull-down assays all BRD4 isoforms interacted with full-length MTHFD1 but not with the dehydrogenase/cyclohydrolase or formyltetrahydrofolate-synthase domains alone (see FIG. 14C). Chromatin extracts comprising tightly DNA-bound proteins from HAP1 cells were prepared and the presence of BRD4 and MTHFD1 was checked by WB. Both proteins were clearly detectable in the chromatin-bound fraction (see FIG. 2C). To probe whether BRD4 recruited MTHFD1 to chromatin, HAP1 cells were treated with small molecule degronimids dBET1⁴⁰ and dBET6⁴¹. Two-hour treatment with these compounds resulted in the near-total ablation of BRD4 from chromatin. Under these conditions MTHFD1 was lost from chromatin, with remaining levels correlating with the amount of chromatin-bound BRD4. Therefore, these data strongly indicate that BRD4 is the sole factor recruiting MTHFD1 to chromatin. The inventors achieved similar results when K562, MOLM-13, MV4-11 and MEG01 cell lines were treated with dBET6 (see FIG. 14D). The chromatin-recruitment of another metabolic enzyme, SHMT1, was only also affected by BRD4 degradation but to a lesser degree. Surprisingly, it was observed that the antifolate MTX caused a similar depletion of chromatin-associated MTHFD1, while it did not affect BRD4 levels. A possible explanation could be a direct competition of the binding between BRD4 and MTHFD1-K56ac, since this key acetylated residue resides inside the putative MTX binding pocket (see FIG. 9J). Importantly, BRD4 degradation was not impairing MTHFD1 (or SHMT1) nuclear localization, neither was MTX treatment (see FIGS. 2C and 2D), indicating that the nuclear import itself is otherwise mediated, while the interaction with BRD4 accounts for the recruitment of MTHFD1 to chromatin.

MTHFD1 Occupies Defined Genomic Loci at a Subset of BRD4 Binding Sites

Having characterized BRD4-dependent chromatin recruitment of MTHFD1, the inventors wanted to map the genomic binding sites of the folate pathway enzyme. Therefore, ChIPmentation experiments¹⁷ were performed in HAP1 cells. MTHFD1 was found to bind to distinct genomic loci and in total 242 MTHFD1 peaks along the genome were observed. The overlap between MTHFD1 binding sites and BRD4 loci was analyzed next. In line with the proteomic experiments, the vast majority of MTHFD1 binding sites overlapped with BRD4 binding sites. MTHFD1 binding sites are predominantly found in proximity of BRD4 peaks (see FIG. 3A). The colocalization between BRD4 and MTHFD1 peaks prevalently happens at promoters and enhancers regions, where also H3K27Ac is enriched (see FIGS. 3B, 12A and 12B), indicating a fundamental role of the folate pathway enzyme promoting transcription. Moreover, MTHFD1 could be found also at intragenic regions, where only a weak amount of BRD4 was present (see FIGS. 10A, 10B and 10C). This evidence indicates that MTHFD1 is needed during the full transcription process and not only to promote its beginning. Moreover, the minimal amount of BRD4 accumulated intragenically is still sufficient to recruit MTHFD1 on the chromatin. Moreover, the inventors performed ChIP-Seq assay in order to further validate the presence on MTHFD1 on chromatin loci occupied by BRD4. MTHFD1 was bound to distinct genomic loci and binding was lost after 2 h treatment with dBET6 (see FIGS. 15A, 16A and 16B). In line with the proteomic experiments, they found that the vast majority of MTHFD1 binding sites overlapped with BRD4 binding sites at promoter and enhancers regions, where also H3K27ac was enriched (see FIGS. 15B, 15C, 15D, 16C and 16D) indicating a widespread role of MTHFD1 in transcriptional control. The inventors also performed transcriptomic analysis and found that in HAP1 cells there was a strong correlation of transcription changes following treatment with BRD4 inhibitors, degraders and antifolates, as well as between knock-down of BRD4 and of MTHFD1 (see FIGS. 15E and 17A). Integration of ChIP-Seq and transcriptomic data showed that both MTHFD1 and BRD4 were enriched at promoters of genes that were downregulated following knock-down of either of these proteins (see FIGS. 15F and 17B). Finally, the inventors could show that the strong correlation between transcriptional effects of BET inhibitors and antifolates, as well as between knock-down of MTHFD1 and BRD4 observed in HAP1 cells, was conserved in K-562 and A549 cells, indicating cell type independence (see FIGS. 18A and 18B).

MTHFD1 and BRD4 Control Nuclear Metabolite Composition

MTHFD1 is a C-1-tetrahydrofolate synthase that catalyzes three enzymatic reactions in folate metabolism, resulting in the interconversion of tetrahydrofolate (THF), 10-formyltetrahydrofolate (10-CHO-THF), 5,10-methenyltetrahydrofolate (5,10-CH=THF) and 5,10-methylenetetrahydrofolate (5,10-CH₂-THF). These folates are key intermediates of one carbon metabolism and provide activated C1 groups for the biosynthesis of purines, pyrimidines and methionine. All three classes of C1 metabolism products have the potential to contribute to transcriptional control. Pyrimidines and purines are incorporated into nucleobases, which in turn are converted in the nucleotides, which are the substrates for the replicative and transcriptional machinery. Methionine metabolism results in the generation of S-Adenosyl-Methionine (SAM), the methyldonor for all histone and DNA-methyltransferases. Biosynthesis of the three major classes of C1 metabolism products, purines, pyrimidines and methionine, is considered to occur in the cytoplasm and mitochondria of mammalian cells. To test whether the entire biosynthetic pathway occurs in the nucleus, the chromatin-associated protein fraction was analyzed for metabolic enzymes. Both thymidylate synthase and several enzymes of the purine biosynthesis pathways (GART, PAICS, ATIC) were found bound to chromatin in HAP1 cells (see FIG. 4A). In contrast, none of the enzymes in methionine and SAM metabolism were detected. These data indicate that potentially the entire purine and pyrimidine biosynthesis occurs also in a chromatin environment. The inventors performed the same experiment in the K-562 cell line and confirmed the presence of enzymes of the pyrimidine and purine biosynthesis pathways on the chromatin fraction (see FIGS. 19A and 20). The inventors therefore asked the question whether inhibition of BRD4 or MTHFD1 altered nuclear metabolite composition. To this aim, they knocked down either BRD4 or MTHFD1, isolated nuclei and analyzed their composition in a targeted metabolomics approach relative to a non-targeting control hairpin (see FIG. 12A). In total, 2851 metabolites were detected, of which 459 were significantly changed in one of the conditions (see FIGS. 4B, 12B and 12C). Interestingly, a surprising correlation was observed between the nuclear metabolomes in BRD4 and MTHFD1 knock-down conditions (see FIG. 4C; correlation coefficient 0.7). The correlation increased considerably when focusing the analysis in the nuclear folate metabolites (see FIG. 4D; correlation coefficient 0.9). Interestingly, among these metabolites, the levels of 10-CHO-THF and 5,10-CH₂-THF, both MTHFD1 direct products, were similarly reduced in MTHFD1 and BRD4 knock-down. In addition, significant changes were detected in purine and pyrimidine metabolites but not methionine derivatives. By both knock-downs, succinyladenosine, N3-hydroxyethylcytosine and thioguanosine-5′-disulfate were reduced, whereas levels of inosine, cytosine, adenosine, AMP, CMP, ADP, isopentenyl adenosine were strongly increased. Part of these changes might be compensatory due to the long treatment time in shRNA experiments. Furthermore, the inventors showed that BET inhibitors and MTX caused highly correlated characteristic changes specifically in the nuclear folate pool that were not observed with other cytotoxic compounds (see FIG. 19B). Overall, a common nuclear metabolite signature for inhibition of the folate biosynthesis and of BRD4 is evident.

BRD4 Inhibitors Synergize with Anti-Folates in Diverse Cancer Cell Lines

Based on the similarities in nuclear metabolite composition following loss of MTHFD1 and BRD4, it was speculated that antifolates might synergize with BRD4 inhibitors in cancer cells. To test this hypothesis, a panel of six cell lines were selected, including four cell lines described to be not sensitive to BRD4 inhibition, plus KBM7 and HAP1 which were routinely used for the experiments (see FIGS. 13A and 13B). Dose response curves confirmed the low sensitivity of these cell lines to (S)-JQ1 treatment and a moderate to low sensitivity to MTX, NOMO-1 being the most sensitive. Despite the poor response to both single treatments, the combination of both drugs efficiently impaired cell viability in all the 6 cell lines tested, at concentrations without any single-agent activity (see FIG. 5A). The calculation of the differential volume (Bliss test⁴²) indicates a strong degree of synergism between the two treatments, validating the hypothesis of a crucial role of nuclear folate metabolite concentration for cell survival. To exclude possible off-target effects of MTX, the inventors treated the cell line showing the strongest drug synergism, A549, with shRNA for MTHFD1 and demonstrated increased sensitivity to (S)-JQ1 (see FIG. 21A). They then proved that BET bromodomain inhibitors can be combined with antifolates in vivo to specifically inhibit cancer cell proliferation without exerting general toxicity. When the inventors treated an A549 xenograft mouse model³⁵ with MTX and (S)-JQ1 alone and in combination, tumor growth was not impaired by either of the individual compounds, but arrested when the two inhibitors were given together (see FIGS. 21B, 21C and 21D). Finally, using two of the reporter cell lines, REDS1 and REDS3, the synergism was shown also at the level of chromatin rearrangement. Indeed, even though the Redness was only weakly increased after three days of MTX treatment (see FIG. 13C), MTX and (S)-JQ1 co-treatment remarkably amplified the basal Redness signal given by (S)-JQ1 alone (see FIG. 5B). This last evidence clearly indicates that the chromatin remodeling process can be enhanced when inhibiting BRD4 and MTHFD1 together, emphasizing the fundamental role of folate metabolites in epigenetic regulation.

### REFERENCES

1. Floyd, S. R. et al. The bromodomain protein Brd4 insulates chromatin from DNA damage signalling. Nature 498, 246-50 (2013),
2. Zuber, J., Shi, J., Wang, E. & Rappaport, A. RNAi screen identifies Brd4 as a therapeutic target in acute myeloid leukaemia. Nature 478, 524-528 (2011).
3. Dey, A., Chitsaz, F., Abbasi, A., Misteli, T. & Ozato, K. The double bromodomain protein Brd4 binds to acetylated chromatin during interphase and mitosis. Proc. Natl. Acad. Sci. U.S.A. 100, 8758-63 (2003).
4. Maruyama, T. et al. A Mammalian Bromodomain Protein, Brd4, Interacts with Replication Factor C and Inhibits Progression to S Phase. 22, 6509-6520 (2002).
5. Yang, Z., He, N. & Zhou, Q. Brd4 recruits P-TEFb to chromosomes at late mitosis to promote G1 gene expression and cell cycle progression. Mol. Cell. Biol. 28, 967-976 (2008).
6. Filippakopoulos, P. et al. Histone recognition and large-scale structural analysis of the human bromodomain family. Cell 149, 214-231 (2012).
7. Bandopadhayay, P. et al. BET Bromodomain Inhibition of MYC-Amplified Medulloblastoma. Clin. Cancer Res. (2014). doi:10.1158/1078-0432.CCR-13-2281
8. Sahni, J. M. et al. Bromodomain and extraterminal protein inhibition blocks growth of triple-negative breast cancers through the suppression of Aurora kinases. J. Biol. Chem. jbc.M116.738666 (2016). doi:10.1074/jbc.M116.738666
9. Rathert, P. et al. Europe PMC Funders Group Transcriptional plasticity promotes primary and acquired resistance to BET inhibition. Nature 525, 543-547 (2016).
10. Fong, C. Y. et al. BET inhibitor resistance emerges from leukaemia stem cells. Nature 525, 538-+ (2015).
11. Ottinger, M. et aL Kaposi's sarcoma-associated herpesvirus LANA-1 interacts with the short variant of BRD4 and releases cells from a BRD4- and BRD2/RING3-induced G1 cell cycle arrest. J. Virol. 80, 10772-10786 (2006).
12. Zhang, Q. et al. Structural Mechanism of Transcriptional Regulator NSD3 Recognition by the ET Domain of BRD4. Structure 24, 1201-1208 (2016).
13. Shen, C. et al. NSD3-Short Is an Adaptor Protein that Couples BRD4 to the CHD8 Chromatin Remodeler. Mol. Cell 60, 847-859 (2015).
14. Rahman, S. et al. The Brd4 extraterminal domain confers transcription activation independent of pTEFb by recruiting multiple proteins, including NSD3. Mol. Cell. Biol. 31, 2641-52 (2011).
15. Sdelci, S. et al. Mapping the chemical chromatin reactivation landscape identifies BRD4-TAF1 cross-talk. (2016), doi:10.1038/nCHeMBIO.2080
16. Carette, J. E. et al. Global gene disruption in human cells to assign genes to phenotypes. nat biotechnol 29, 542-546 (2011).
17. Schmidl, C., Rendeiro, A. F., Sheffield, N. C. & Bock, C. ChIPmentation: fast, robust, low-input ChIP-seq for histones and transcription factors. Nat. Methods 12, 963-5 (2015).
18. Kim, T. H. et al. A high-resolution map of active promoters in the human genome. Nature 436, 876-880 (2007).
19. Jiang, H., Lei, R., Ding, S.-W. & Zhu, S. Skewer: a fast and accurate adapter trimmer for next-generation sequencing paired-end reads. BMC Bioinformatics 15, 182 (2014),
20. Langmead, B. & Steven L., S. Fast gapped-read alignment with Bowtie 2. Nat. Methods 9, 357-359 (2013).
21. Landt, S. & Marinov, G. ChIP-seq guidelines and practices of the ENCODE and modENCODE consortia. Genome . . . 1813-1831 (2012), doi:10.1101/gr.136184.111.
22. Quinlan, A. R. BEDTools: The Swiss-Army tool for genome feature analysis. Current Protocols in Bioinformatics 2014, (2014).
23. Robinson, J. T. et al. Integrative Genome Viewer. Nat. Biotechnol. 29, 24-6 (2011).
24. Sven, H. et al. Simple combinations of lineage-determining transcription factors prime cis-regulatory elements required for macrophage and B cell identities. Mol. Cell 38, 576-589 (2010).
25. Ross-Innes, C. S. et al. Differential oestrogen receptor binding is associated with clinical outcome in breast cancer. Nature 481, 389-393 (2012).
26. Stempor, P. & Ahringer, J. SeqPlots—Interactive software for exploratory data analyses, pattern discovery and visualization in genomics. Wellcome Open Res. 1, 14 (2016).
27. Chen, E. Y. et al. Enrichr: interactive and collaborative HTML5 gene list enrichment analysis tool. BMC Bioinformatics 14, 128 (2013).
28. Berman, H. M. et al. The Protein Data Bank. Nucleic Acids Res. 28, 235-242 (2000).
29. Dutta, B. et al. Elucidating the temporal dynamics of chromatin-associated protein release upon DNA digestion by quantitative proteomic approach. J. Proteomics 75, 5493-5506 (2012).
30. Zougman, A., Nagaraj, N., Mann, M. & Wisniewski, J. R. Universal sample preparation method for proteome analysis. Nat. Methods 6, 359-62 (2009).
31. Maurer, M. et al. Comprehensive comparative and semiquantitative proteome of a very low number of native and matched Epstein-Barr-Virus-transformed B lymphocytes infiltrating human melanoma. J. Proteome Res. 13, 2830-2845 (2014).
32. Breitwieser, F. P. et al. General statistical modeling of data from protein relative expression isobaric tags. J. Proteome Res. 10, 2758-2766 (2011).
33. Xia, J. & Wishart, D. S. MSEA: A web-based tool to identify biologically meaningful patterns in quantitative metabolomic data. Nucleic Acids Res. 38, 71-77 (2010).
34. Xia, J., Sinelnikov, I. V., Han, B. & Wishart, D. S. MetaboAnalyst 3.0-making metabolomics more meaningful. Nucleic Acids Res. 43, W251-W257 (2015).
35. Grabner, B. et al. Disruption of STAT3 signalling promotes KRAS-induced lung tumorigenesis. Nat. Commun. 6, 6285 (2015).
36. Anderson, D. D. & Stover, P. J. SHMT1 and SHMT2 are functionally redundant in nuclear de novo thymidylate biosynthesis. PLoS One 4, (2009).
37. Anderson, D. D., Woeller, C. F. & Stover, P. J. Small ubiquitin-like modifier-1 (SUMO-1) modification of thymidylate synthase and dihydrofolate reductase. Clin. Chem. Lab. Med. 45, 1760-1763 (2007).
38. Woeller, C. F., Anderson, D. D., Szebenyi, D. M. E. & Stover, P. J. Evidence for small ubiquitin-like modifier-dependent nuclear import of the thymidylate biosynthesis pathway. J. Biol. Chem. 282, 17623-17631 (2007).
39. Field, M. S., Kamynina, E. & Stover, P. J. MTHFD1 Regulates Nuclear de novo Thymidylate Biosynthesis and Genome Stability. Biochimie 27-30 (2016). doi:10.1007/128
40. Winter, G. E., Buckley, D, L. & Bradner, J. E. Selective Target Protein Degradation via Phtalimide Conjugation. Science 348, 37-54 (2015).
41. Winter, G. E. et al. BET Bromodomain Proteins Function as Master Transcription Elongation Factors Independent of CDK9 Recruitment. Mol. Cell 67, 5-18.e19 (2017).
42. Bliss, C. I. The toxicity of poisons applied jointly. Ann. Appl. Biol. 26, 585-615 (1939).

1. A BRD4 inhibitor for use in treating or preventing cancer, wherein the BRD4 inhibitor is to be administered in combination with an antifolate.

2. An antifolate for use in treating or preventing cancer, wherein the antifolate is to be administered in combination with a BRD4 inhibitor.

3. A combination of a BRD4 inhibitor and an antifolate or use in treating or preventing cancer.

4. A pharmaceutical composition comprising a BRD4 inhibitor, an antifolate, and a pharmaceutically acceptable excipient.

5. The pharmaceutical composition of claim 4 for use in treating or preventing cancer.

6. The BRD4 inhibitor for use according to claim 1 or the antifolate for use according to claim 2 or the combination for use according to claim 3 or the pharmaceutical composition for use according to claim 5, wherein said cancer is a BRD4 inhibitor-resistant cancer.

7. An antifolate for use in resensitizing a BRD4 inhibitor-resistant cancer to the treatment with a BRD4 inhibitor.

8. The BRD4 inhibitor for use according to claim 1 or 6 or the antifolate for use according to claim 2 or 6 or the combination for use according to claim 3 or 6 or the pharmaceutical composition for use according to claim 5 or 6 or the antifolate for use according to claim 7, wherein said cancer is selected from prostate cancer, breast cancer, acute myeloid leukemia, acute lymphocytic leukemia, non-Hodgkin's lymphoma, multiple myeloma, bladder cancer, head and neck cancer, glioblastoma, mesothelioma, osteogenic sarcoma, choriocarcinoma, and NUT midline carcinoma.

9. The BRD4 inhibitor for use according to claim 1, 6 or 8 or the antifolate for use according to claim 2, 6 or 8 or the combination for use according to claim 3, 6 or 8 or the pharmaceutical composition of claim 4 or the pharmaceutical composition for use according to claim 5, 6 or 8 or the antifolate for use according to claim 7 or 8, wherein the BRD4 inhibitor is (S)-JQ1, CeMMEC2, I-BET 151, I-BET 762, PF-1, bromosporine, OTX-015, TEN-010, CPI-203, CPI-0610, RVX-208, BI2536, TG101348, LY294002, or a pharmaceutically acceptable salt or solvate of any of these agents.

10. The BRD4 inhibitor for use according to claim 9 or the antifolate for use according to claim 9 or the combination for use according to claim 9 or the pharmaceutical composition of claim 9 or the pharmaceutical composition for use according to claim 9 or the antifolate for use according to claim 9, wherein the BRD4 inhibitor is (S)-JQ1.

11. The BRD4 inhibitor for use according to any one of claims 1, 6 and 8 to 10 or the antifolate for use according to any one of claim 2, 6 and 8 to 10 or the combination for use according to any one of claims 3, 6 and 8 to 10 or the pharmaceutical composition of claim 4, 9 or 10 or the pharmaceutical composition for use according to any one of claims 5, 6 and 8 to 10 or the antifolate for use according to any one of claims 7 to 10, wherein the antifolate is an MTHFD1 inhibitor.

12. The BRD4 inhibitor for use according to any one of claims 1, 6 and 8 to 11 or the antifolate for use according to any one of claim 2, 6 and 8 to 11 or the combination for use according to any one of claims 3, 6 and 8 to 11 or the pharmaceutical composition of any one of claims 4 and 9 to 11 or the pharmaceutical composition for use according to any one of claims 5, 6 and 8 to 11 or the antifolate for use according to any one of claims 7 to 11, wherein the antifolate is methotrexate, pemetrexed, trimetrexate, edatrexate, lometrexol, 5-fluorouracil, pralatrexate, aminopterin, or a pharmaceutically acceptable salt or solvate of any of these agents.

13. The BRD4 inhibitor for use according to claim 12 or the antifolate for use according to claim 12 or the combination for use according to claim 12 or the pharmaceutical composition of claim 12 or the pharmaceutical composition for use according to claim 12 or the antifolate for use according to claim 12, wherein the antifolate is methotrexate or a pharmaceutically acceptable salt or solvate thereof.

14. The BRD4 inhibitor for use according to claim 1, 6 or 8 or the antifolate for use according to claim 2, 6 or 8 or the combination for use according to claim 3, 6 or 8 or the pharmaceutical composition of claim 4 or the pharmaceutical composition for use according to claim 5, 6 or 8 or the antifolate for use according to claim 7 or 8, wherein the BRD4 inhibitor is (S)-JQ1, and wherein the antifolate is methotrexate or a pharmaceutically acceptable salt or solvate thereof.

15. A method of assessing the susceptibility or responsiveness of a subject to the treatment with a BRD4 inhibitor, wherein the subject has been diagnosed as suffering from cancer or is suspected of suffering from cancer, the method comprising determining the level of nuclear folate and/or the level of expression of MTHFD1 in a sample obtained from the subject.

16. A method of assessing the susceptibility or responsiveness of a subject to the treatment with a BRD4 inhibitor, wherein the subject has been diagnosed as suffering from cancer or is suspected of suffering from cancer,
the method comprising a step of determining the level of nuclear folate and/or the level of expression of MTHFD1 in a sample obtained from the subject,
wherein a smaller level of nuclear folate and/or a smaller expression level of MTHFD1 in the sample from the subject is/are indicative of the subject being more susceptible or more responsive to the treatment with a BRD4 inhibitor.

17. A method of assessing the susceptibility or responsiveness of a subject to the treatment with a BRD4 inhibitor, wherein the subject has been diagnosed as suffering from cancer or is suspected of suffering from cancer,
the method comprising a step of determining the level of nuclear folate in a sample obtained from the subject,
wherein a smaller level of nuclear folate in the sample from the subject is indicative of the subject being more susceptible or more responsive to the treatment with a BRD4 inhibitor.

18. A method of assessing the susceptibility or responsiveness of a subject to the treatment with a BRD4 inhibitor, wherein the subject has been diagnosed as suffering from cancer or is suspected of suffering from cancer,
the method comprising a step of determining the level of expression of MTHFD1 in a sample obtained from the subject,
wherein a smaller expression level of MTHFD1 in the sample from the subject is indicative of the subject being more susceptible or more responsive to the treatment with a BRD4 inhibitor.

19. The method of any one of claims 15 to 18, wherein said cancer is selected from prostate cancer, breast cancer, bladder cancer, head and neck cancer, glioblastoma, mesothelioma, osteogenic sarcoma, choriocarcinoma, and NUT midline carcinoma.

20. The method of any one of claims 15 to 19, wherein the sample is a cancer tissue biopsy sample.

21. The method of any one of claims 15 to 18, wherein said cancer is selected from acute myeloid leukemia, acute lymphocytic leukemia, non-Hodgkin's lymphoma, and multiple myeloma.

22. The method of claim 21, wherein the sample is a blood sample.

23. The method of claim 15, 16 or 18 or any one of their dependent claims 19 to 22, wherein the level of expression of MTHFD1 is determined by determining the level of translation of MTHFD1,
wherein the level of translation is preferably determined using an antibody-based assay, mass spectrometry, a gel-based or blot-based assay, or flow cytometry, more preferably using an immunohistochemical method, an enzyme-linked immunosorbent assay, or a radioimmunoassay.

24. The method of claim 15, 16 or 18 or any one of their dependent claims 19 to 22, wherein the level of expression of MTHFD1 is determined by determining the level of nuclear MTHFD1 protein,
wherein the level of nuclear MTHFD1 protein is preferably determined using an antibody-based assay, more preferably using immunofluorescence staining or an immunohistochemical method.

25. The method of claim 15, 16 or 18 or any one of their dependent claims 19 to 22, wherein the level of expression of MTHFD1 is determined by determining the level of transcription of MTHFD1,
wherein the level of transcription is preferably determined using a quantitative reverse transcriptase polymerase chain reaction or a microarray.

26. The method of any one of claims 15 to 25, wherein the subject is a human.

27. A BRD4 inhibitor for use in the treatment of cancer in a subject, wherein the subject has been identified in the method of any one of claims 15 to 26 as being susceptible or responsive to the treatment with a BRD4 inhibitor.

28. Use of a pair of primers for a transcript of the gene MTHFD1 in a method of assessing the susceptibility or responsiveness of a subject to the treatment with a BRD4 inhibitor, wherein the subject has been diagnosed as suffering from cancer or is suspected of suffering from cancer.

29. Use of a nucleic acid probe to a transcript of the gene MTHFD1 in a method of assessing the susceptibility or responsiveness of a subject to the treatment with a BRD4 inhibitor, wherein the subject has been diagnosed as suffering from cancer or is suspected of suffering from cancer.

30. Use of a microarray comprising a nucleic acid probe to the transcript of the gene MTHFD1 in a method of assessing the susceptibility or responsiveness of a subject to the treatment with a BRD4 inhibitor, wherein the subject has been diagnosed as suffering from cancer or is suspected of suffering from cancer.

31. Use of an antibody against the protein MTHFD1 in a method of assessing the susceptibility or responsiveness of a subject to the treatment with a BRD4 inhibitor, wherein the subject has been diagnosed as suffering from cancer or is suspected of suffering from cancer.

32. The use of any one of claims 28 to 31, wherein said method of assessing the susceptibility or responsiveness of a subject to the treatment with a BRD4 inhibitor is a method as defined in claim 15, 16 or 18 or any one of their dependent claims 19 to 26.

33. A BRD4 inhibitor for use in a method of treating cancer in a subject that has been diagnosed as suffering from cancer or is suspected of suffering from cancer, the method comprising:
determining the level of nuclear folate and/or the level of expression of MTHFD1 in a sample obtained from the subject;
determining whether or not the subject is susceptible or responsive to the treatment with a BRD4 inhibitor, wherein a smaller level of nuclear folate and/or a smaller expression level of MTHFD1 in the sample from the subject is/are indicative of the subject being more susceptible or more responsive to the treatment with a BRD4 inhibitor; and
administering a BRD4 inhibitor to the subject if the subject has been identified as being susceptible or responsive to the treatment with a BRD4 inhibitor.

34. A BRD4 inhibitor for use in a method of treating cancer in a subject that has been diagnosed as suffering from cancer or is suspected of suffering from cancer, the method comprising;
determining the level of nuclear folate in a sample obtained from the subject;
determining whether or not the subject is susceptible or responsive to the treatment with a BRD4 inhibitor, wherein a smaller level of nuclear folate in the sample from the subject is indicative of the subject being more susceptible or more responsive to the treatment with a BRD4 inhibitor; and
administering a BRD4 inhibitor to the subject if the subject has been identified as being susceptible or responsive to the treatment with a BRD4 inhibitor.

35. A BRD4 inhibitor for use in a method of treating cancer in a subject that has been diagnosed as suffering from cancer or is suspected of suffering from cancer, the method comprising:
determining the level of expression of MTHFD1 in a sample obtained from the subject;
determining whether or not the subject is susceptible or responsive to the treatment with a BRD4 inhibitor, wherein a smaller expression level of MTHFD1 in the sample from the subject is indicative of the subject being more susceptible or more responsive to the treatment with a BRD4 inhibitor; and
administering a BRD4 inhibitor to the subject if the subject has been identified as being susceptible or responsive to the treatment with a BRD4 inhibitor.

36. A BRD4 inhibitor for use in a method of treating cancer in a subject that has been diagnosed as suffering from cancer or is suspected of suffering from cancer, the method comprising:
determining the level of nuclear MTHFD1 protein in a sample obtained from the subject;
determining whether or not the subject is susceptible or responsive to the treatment with a BRD4 inhibitor, wherein a smaller level of nuclear MTHFD1 protein in the sample from the subject is indicative of the subject being more susceptible or more responsive to the treatment with a BRD4 inhibitor; and
administering a BRD4 inhibitor to the subject if the subject has been identified as being susceptible or responsive to the treatment with a BRD4 inhibitor.

37. The BRD4 inhibitor for use according to any one of claims 33 to 36, wherein said cancer is selected from prostate cancer, breast cancer, bladder cancer, head and neck cancer, glioblastoma, mesothelioma, osteogenic sarcoma, choriocarcinoma, and NUT midline carcinoma.

38. The BRD4 inhibitor for use according to any one of claims 33 to 37, wherein the sample is a cancer tissue biopsy sample.

39. The BRD4 inhibitor for use according to any one of claims 33 to 36, wherein said cancer is selected from acute myeloid leukemia, acute lymphocytic leukemia, non-Hodgkin's lymphoma, and multiple myeloma.

40. The BRD4 inhibitor for use according to claim 39, wherein the sample is a blood sample.

41. The BRD4 inhibitor for use according to claim 33 or 35 or any one of their dependent claims 37 to 40, wherein the level of expression of MTHFD1 is determined by determining the level of translation of MTHFD1,
wherein the level of translation is preferably determined using an antibody-based assay, mass spectrometry, a gel-based or blot-based assay, or flow cytometry, more preferably using an immunohistochemical method, an enzyme-linked immunosorbent assay, or a radioimmunoassay.

42. The BRD4 inhibitor for use according to claim 33 or 35 or any one of their dependent claims 37 to 40, wherein the level of expression of MTHFD1 is determined by determining the level of transcription of MTHFD1,
wherein the level of transcription is preferably determined using a quantitative reverse transcriptase polymerase chain reaction or a microarray.

43. The BRD4 inhibitor for use according to claim 33 or 35 or any one of their dependent claims 37 to 40, wherein the level of expression of MTHFD1 is determined by determining the level of nuclear MTHFD1 protein,
wherein the level of nuclear MTHFD1 protein is preferably determined using an antibody-based assay, more preferably using immunofluorescence staining or an immunohistochemical method.

44. The BRD4 inhibitor for use according to any one of claims 33 to 43, wherein the subject is a human.

45. The method of any one of claims 15 to 26 or the BRD4 inhibitor for use according to claim 27 or the use of any one of claims 28 to 32 or the BRD4 inhibitor for use according to any one of claims 33 to 44, wherein the BRD4 inhibitor is (S)-JQ1, CeMMEC2, I-BET 151, I-BET 762, PF-1, bromosporine, OTX-015, TEN-010, CPI-203, CPI-0610, RVX-208, BI2536, TG101348, LY294002, or a pharmaceutically acceptable salt or solvate of any of these agents.
